(19) United States
(12) Patent Application Publication
(10) Pub. No.: US 2026/0335294 A1
(43) Pub. Date: Oct. 29, 2026

(54) TREATING CONGENITAL DISORDERS OF GLYCOSYLATION

(71) Applicants: Mayo Foundation for Medical Education and Research, Rochester, MN (US); VIB vzw, Gent (BE); Katholieke Universiteit Leuven, Leuven (BE)

(72) Inventors: Eva Morava-Kozicz, Rochester, MN (US); Laszlo Tamás Kozicz, New York, NY (US); Silvia Radenkovic, Rochester, MN (US); Bart Ghesquière, Leuven (BE)

(21) Appl. No.: 19/113,522

(22) PCT Filed: Sep. 19, 2023
(86) PCT No.: PCT/US2023/033155

§ 371 Date: Mar. 20, 2025

Related U.S. Application Data
(60) Provisional application No. 63/408,299, filed on Sep. 20, 2022.

(51) Int. Cl. A61K 31/426; A61K 31/708; A61P 3/00

(52) U.S. Cl. CPC A61K 31/426; A61K 31/708; A61P 3/00

(57) ABSTRACT

This document relates to methods and materials for treating congenital disorders of glycosylation (CDG; e.g., a phosphomannomutase 2-congenital disorder of glycosylation (PMM2-CDG)). For example, this document provides methods and materials for using (a) one or more aldose reductase (AR) inhibitors (e.g., epalrestat) and (b) one or more nucleosides to treat a mammal having one or more CDG (e.g., PMM2-CDG).

### CROSS-REFERENCE TO RELATED APPLICATIONS

This application claims the benefit of U.S. Patent Application Ser. No. 63/408,299, filed on Sep. 20, 2022. The disclosure of the prior application is considered part of, and is incorporated by reference in, the disclosure of this application.

### TECHNICAL FIELD

This document relates to methods and materials for treating congenital disorders of glycosylation (CDG; e.g., a phosphomannomutase 2-congenital disorder of glycosylation (PMM2-CDG)). For example, this document provides methods and materials for using one or more aldose reductase (AR) inhibitors (e.g., epalrestat) in combination with one or more nucleosides (e.g., guanosine, uridine, adenosine, thymidine, and/or cytidine) to treat a mammal having one or more CDG (e.g., PMM2-CDG).

### BACKGROUND INFORMATION

CDG are a rapidly growing group of inherited metabolic disorders caused by pathogenic variants in genes coding for the enzymes and transporters affecting glycosylation. There are more than 160 CDG described to date (Boyer et al., Trends Mol. Med., 28:463-481 (2022); Ferreira et al., J. Inherit. Metab. Dis., 44:164-177 (2021); and Freeze et al., Biochim. Biophys. Acta, 1792(9):835-840 (2022)). The most common CDG is PMM2-CDG, with more than 1000 reported patients and an estimated prevalence of 1:18,745 (Non-Finish Europeans)-1:366,999 (South Asian population) (Pajusalu et al., Front. Gen. 12: 719437 (2021)). PMM2-CDG is caused by a deficiency in the PMM2 enzyme (Schaftingen and Jaeken, FEBS Lett., 377(3):318-20 (1995)), which leads to the depletion of mannose 1-phosphate (man-1-P) and its downstream metabolite guanosine diphosphate (GDP)-mannose (Körner et al., Glycobiology, 8:165-171 (1998)). There is currently no known cure for PMM2-CDG (Altassan et al., J. Inherit. Metab. Dis., 42:5-28 (2019)).

### SUMMARY

This document provides methods and materials for treating a mammal (e.g., a human such as a juvenile human) having one or more CDG (e.g., PMM2-CDG). For example, (a) one or more AR inhibitors (e.g., epalrestat) and (b) one or more nucleosides (e.g., guanosine, uridine, adenosine, thymidine, and/or cytidine) can be administered to a mammal having one or more CDG (e.g., PMM2-CDG) to treat the mammal. As demonstrated herein, administering (a) one or more AR inhibitors (e.g., epalrestat) and (b) one or more nucleosides (e.g., guanosine, uridine, adenosine, thymidine, and/or cytidine) to a mammal (e.g., a human such as a juvenile human) having one or more CDG (e.g., PMM2-CDG) can increase a level of GDP-mannose within the mammal. Having the ability to restore GDP-mannose levels within a mammal as described herein (e.g., by administering one or more AR inhibitors (e.g., epalrestat) and one or more nucleosides (e.g., guanosine) and/or one or more nucleotides (e.g., guanosine monophosphate, guanosine diphosphate, and/or guanosine triphosphate)) provides a unique and unrealized opportunity to treat mammals (e.g., humans such as a juvenile humans) having one or more CDG (e.g., PMM2-CDG).

In general, one aspect of this document features methods for treating a mammal having a CDG The methods can include, or consist essentially of, administering, to a mammal having a CDG, (a) one or more aldose reductase (AR) inhibitors and (b) one or more nucleosides and/or one or more nucleotides, where one or more symptoms of the CDG are reduced. The mammal can be a human. The human can be a juvenile human. The human can be from about 1 month to about 17 years of age. The mammal can be identified as having the CDG The CDG is a phosphomannomutase 2 (PMM2)-CDG The one or more AR inhibitors can be epalrestat, AT007, fidarestat, sorbinil, salfredin b11, or any combination thereof. The one or more nucleosides can be guanosine, uridine, adenosine, thymidine, cytidine, or any combination thereof. The method can include administering epalrestat and guanosine. The one or more AR inhibitors and the one or more nucleosides can be administered to the mammal by oral administration. The one or more AR inhibitors and said the or more nucleosides can be administered to the mammal simultaneously. The one or more AR inhibitors and the one or more nucleosides can be administered to the mammal as a single composition. The one or more AR inhibitors and the one or more nucleosides can be administered to the mammal separately. The one or more symptoms can include ataxia, speech delay, seizures, global developmental delay, intellectual disability, coagulation abnormalities, or liver abnormalities.

In another aspect, this document features methods for increasing a level of one or more PMM2-related metabolites within a mammal. The methods can include, or consist essentially of, administering, to a mammal, (a) one or more AR inhibitors and (b) one or more nucleosides and/or one or more nucleotides. The mammal can be a human. The human can be a juvenile human. The human can be from about 1 month to about 17 years of age. The mammal can have a CDG. The CDG can be a PMM2-CDG. The one or more AR inhibitors can be epalrestat, AT007, fidarestat, sorbinil, salfredin b11, or any combination thereof. The one or more nucleosides can be guanosine, uridine, adenosine, thymidine, cytidine, or any combination thereof. The method can include administering epalrestat and guanosine to the mammal. The one or more AR inhibitors and the one or more nucleosides can be administered to the mammal by oral administration. The one or more AR inhibitors and the one or more nucleosides can be administered to the mammal simultaneously. The one or more AR inhibitors and the one or more nucleosides can be administered to the mammal as a single composition. The one or more AR inhibitors and the one or more nucleosides can be administered to the mammal separately. The one or more PMM2-related metabolites can be mannose-1-phophate (man-1-P), mannose-6-phosphate (man-6-P), glucose-1,6-diphosphate (Glc-1,6-P2), guanosine, uridine, adenosine, thymidine, cytidine, guanosine triphosphate (GTP), guanosine diphosphate (GDP), GDP-mannose, GDP-fucose, UDP-hexose, UDP-HexNAc, or any combinations thereof.

In another aspect, this document features uses of a composition including (a) one or more AR inhibitors and (b) one or more nucleosides, to treat a mammal having a CDG

In another aspect, this document features compositions including (a) one or more AR inhibitors and (b) one or more nucleosides and/or one or more nucleotides, for use in the preparation of a medicament to treat a mammal having a CDG

In another aspect, this document features compositions including (a) one or more AR inhibitors and (b) one or more nucleosides and/or one or more nucleotides, for use in the treatment of a mammal having a CDG

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1E. Disturbed polyol metabolism is a hallmark of PMM2-CDG. FIG. 19A) Urine sorbitol and mannitol were increased in PMM2-CDG G. 1A) Urine sorbitol and mannitol were increased in PMM2-CDG. Urine sorbitol and mannitol were measured by GC/MS in PMM2-CDG patients (n=50). Higher limit of the control reference range (n=533) is given on the left. (Control reference range values sorbitol<5 mmol/mol creatine; mannitol 20 mmol/mol creatine). FIG. 1B) Heatmap of the 25 top discriminating intracellular metabolites evaluated by LC/MS. Metabolomics analysis showed PMM2-CDG fibroblasts clustered separately from healthy control due to significant differences in relative abundances of multiple metabolites in PMM2 deficient fibroblasts compared to healthy controls. Clustering result shown as heatmap (distance measured using euclidean, and clustering algorithm using ward. PMM2-CDG (n=6, t=1-8) and healthy control fibroblasts (n=5, t=1-6). FIG. 1C) Man-1-P and GDP-mannose were depleted in PMM2-CDG fibroblasts while intracellular polyols (sorbitol and mannitol) were increased. Man-1-P, sorbitol, and mannitol were measured by GC/MS; GDP-mannose and polyol pool was measured by LC/MS (PMM2-CDG n=6, t=1-8; Control n=5, t=1-6). FIG. 1D Nucleotides are depleted in PMM2-CDG. Nucleotides were measured by LC/MS. PMM2-CDG n=6, t=1-8; Control n=5, t=1-6). FIG. 1E) Zebrafish model of PMM2-CDG showed similar metabolic changes to PMM2-CDG fibroblasts. GDP-mannose was significantly lower in pmm2 mutant zebrafish compared to the wild-type (wt) controls, while polyols were increased (pmm2 mutant n=4, t=1; wt n=3, t=1; each metabolomic sample was generated from a pool of 10 zebrafish).

PMM enzymatic activity measured in pmm2 mutant zebrafish. FIG. 1D) Average pmm activity of wt is given on the left. PMM enzymatic activity was measured in three different samples each containing 10 pmm2 mutant zebrafish. FIG. 1F) PMM2-CDG affected multiple biochemical pathways. Representation of main findings identified by metabolomic profiling of PMM2-CDG fibroblasts and zebrafish. Means are represented with SD. Student t-test was performed. Abbreviations: CTR=healthy control; wt=wild-type; P=phosphate; P1-6=patient 1-6.

FIGS. 2A-2C. The effect of environment on polyol levels. FIG. 2A) Nondialyzed FBS contains high amounts of mannitol and sorbitol. Sorbitol and mannitol were measured in medium containing dialyzed or non-dialyzed FBS by GC/MS. FIG. 3B) Urine polyols increased upon fructose tolerance test. Urine polyols were collected from P5 before and after fructose administration. FIG. 2C) Fructose availability increased intracellular polyol pool. Abundance (represented by the size of the donut) and fractional contribution of ¹³C₆ glucose/¹³C₆ fructose (represented by the blue/orange color of the donut) of polyol and hexose control (CTR) n=1, t=1. Abbreviations: GLC=glucose; FRC=fructose; CR=creatinine; FC=fractional contribution; FBS=fetal bovine serum. *note: hexose pool measured by LC/MS contains glucose, fructose, mannose and galactose.

FIGS. 3A-3C. Aldose reductase inhibition with epalrestat decreased polyol levels and elicited global metabolic changes in both PMM2-CDG and healthy controls. PMM2-CDG and healthy fibroblasts grown in medium containing dialyzed FBS were treated daily with 10 μM epalrestat. FIG. 3A) Epalrestat decreased intracellular polyol levels. Specifically, polyol pools and sorbitol were significantly decreased upon epalrestat treatment. Polyol—measured by LC/MS. Sorbitol and mannitol—measured by GC/MS. (PMM2-CDG n=6, t=2-4; Healthy control n=5; t=1-4). FIG. 3B) Metabolite abundances linked to PMM2 enzyme were increased upon epalrestat supplementation. Epalrestat was able to significantly increase the abundances of man-1-P, man-6-P, and GDP-mannose as well as Glc-1,6-P2 in patient fibroblasts. PMM2-CDG n=6, t=2-4; Healthy control n=5; t=1-4). FIG. 3C) Pmm2 mutant zebrafish showed increased GDP-mannose and decreased polyol levels after epalrestat treatment. Pmm2 mutant (n=10*, t=1) and wildtype (n=10*, t=1) zebrafish were treated with 10 μM or 40 M epalrestat for 24 hours. (*each zebrafish metabolomics sample represents a pool of 10 zebrafish). Means are represented with SD (where applicable). Two-way ANOVA with repeated measures was used for statistical analysis. For additional metabolites see FIG. 4. Abbreviation: CTR=control; P=patient; wt=wild type; g=p-value reflecting effect of genotype; t=p-value reflecting the effect of the treatment; I=p-value reflecting interaction between treatment and genotype.

FIGS. 4A-4E. Epalrestat supplementation leads to an increase in several metabolites across multiple biochemical pathways. FIG. 4A) AR inhibition resulted in an increase in NADPH/NADP. NADPH/NADP ratio increases after epalrestat treatment. NADP abundance was decreased, while NADPH abundance increased after epalrestat treatment. PMM2-CDG n=3, healthy control n=2, t=1. Two-way repeated measures ANOVA was used for statistical analysis. FIG. 4B) Epalrestat increased abundances of hexose-P (Hex-P), glucose-1-P, and UDP-dexose; FIG. 4C) Epalrestat increased metabolites of hexosamine biosynthesis pathway hexosamine-P, UDP-HexNAc, CMP-sialic acid; and FIG. 4D) pentose phosphate pathway metabolites pentose-P (pool of ribose-5-P, ribose-1-P, etc.); sedoheptulose-7-P. PMM2-CDG n=6, t=2-4; healthy control n=5, t=1-4. FIG. 4E) Polyol pathway metabolites. Fructose and glucose PMM2-CDG n=6, t=1-2; healthy control n=4, t=1-2. Two-way repeated measures ANOVA was used for statistical analysis. Abbreviations: AR=aldose reductase; CTR=control; P1-6=Patient 1-6; P=phosphate; g=p-value reflecting effect of genotype; t=p-value reflecting the effect of the epalrestat treatment; i=p-value reflecting interaction between treatment and genotype.

FIGS. 5A-5C. Effects of aldose inhibition are corroborated in vivo in pmm2 mutant zebrafish. FIG. 5A) Schematic representation of imaging and treatment of zebrafish. Briefly, 5 dpf wt and pmm2 embryos were placed one per well in a 12-well culture dish and treated daily with either 20 M epalrestat in DMSO or DMSO only, refreshing the media daily. After a 15-minute sensory deprivation, locomotor activity was monitored for ten-minute intervals each day (6-10 dpf) using the Zebrabox System. FIG. 5B) Track of swim paths of wt and pmm2 zebrafish with and without epalrestat treatment on 6 dpf and 8 dpf. Slow speed is indicated in green, while fast speed is indicated in red. FIG. 5C) Epalrestat increases swimming distance (mm). Epalrestat significantly increased swimming distance at 6 dpf and 7 dpf in pmm2 zebrafish. (wt n=42-46, pmm2 n=22-51). 5D) Epalrestat increased number of initiated swim events. Epalrestat significantly increased the number of initiated swim events. Mixed effect analysis with repeated measures was used to statistically evaluate data. Means are represented with SD. Abbreviations: dpf=days post fertilization; e=p-value corresponding to the effect of epalrestat treatment; lt=p value corresponding to the length of treatment; g=p-value corresponding to the effect of genotype.

FIGS. 6A-6C. Treatment with nucleosides, especially guanosine, increased GDP-mannose abundance in PMM2-CDG. FIG. 6A) Combination treatment of nucleosides and epalrestat treatment increased GPD-mannose and GDP-fucose abundances. Patient and control fibroblasts were supplemented with nucleotides (30 M each) and 10 μM epalrestat. It was found that the abundance of GDP-mannose and GDP-fucose further increased after the combination therapy of epalrestat and nucleosides compared to epalrestat alone (FIG. 3). (PMM2-CDG n=3, t=1; CTR n=2, t=1). FIG. 6B) Combination treatment of uridine and epalrestat increased GDP-sugar nucleotides. 30 μM uridine was supplemented in combination with epalrestat and resulted in a significant increase of GDP-mannose and GDP-fucose. (PMM2-CDG n=2, t=1, CTR n=2, t=1). FIG. 6C) Combination treatment of guanosine and epalrestat treatment increased GPD-sugar nucleotides to the levels of healthy controls. 30 M guanosine was supplemented in combination with epalrestat and resulted in an increase of GDP-mannose and GDP-fucose to the levels seen in healthy controls. (PMM2-CDG n=2, t=1; CTR n=2, t=1). Two-way repeated measures ANOVA was used for statistical analysis. Abbreviations: CTR=control; t=p-value reflecting effect of treatment; i=p-value reflecting the interaction of genotype and treatment.

FIG. 7. The effects of epalrestat, nucleosides, and the combination treatment on intracellular levels of nucleotides, nucleotide monophosphates and aspartate in PMM2-CDG and healthy control fibroblasts. PMM2-CDG n=3, t=1; healthy control n=2, t=1.

FIG. 8. The effects of epalrestat, nucleosides, and the combination treatment on abundance of other metabolites in PMM2-CDG and healthy control fibroblasts. PMM2-CDG n=3, t=1; healthy control n=2, t=1. Means are represented with SD.

FIGS. 9A-9D. Tracer studies with ¹³C₆ glucose upon AKR1B1 siRNA inhibition reveal metabolic rewiring and increased de novo sugar nucleotide synthesis. FIG. 9A) Methodology. Cells were incubated with either siRNA targeting the aldose reductase gene AKR1B1 or non-targeting (negative) siRNA for 48 hours. Then, medium was changed and the medium containing either ¹³C₆-glucose (tracer glucose) or ¹²C₆-glucose was added. Tracer was then metabolized throughout subsequent biochemical pathways. Metabolites were extracted for metabolomics analysis. Furthermore, membrane bound sialic acid, the end sugar of glycan chains, was isolated and subjected to metabolomics analysis. FIG. 9B) AKR1B1 knock down confirmation. Polyol abundances and FC of ¹³C₆ glucose in polyol were decreased following AR KD. (PMM2-CDG n=2, t=2; healthy control n=2, t=1-2). FIG. 9C) Deconvolution of positional labeling of ¹³C₆ glucose in CMP-sialic acid. Increase in m11, m14, and m16 after AR inhibition and incubation with ¹³C₆ glucose in CMP-sialic acid indicates multiple pathways such as PPP, nucleotide biosynthesis, glucosamine biosynthesis and glycolysis are simultaneously upregulated upon AR inhibition. FIG. 9D) Deconvolution of positional labeling of ¹³C₆ glucose in UDP-HexNAc, GDP-Fucose, CMP-sialic acid FIG. 9E) Increased glucose flux is observed in membrane derived sialic acid, terminal glycan sugar. Increase in FC of ¹³C₆-glucose in sialic acid derived from membranes suggests that sialylation and overall glycosylation is improved upon AR inhibition. Two-way repeated measures ANOVA (FIG. 9B and FIG. 9C) and mixed effect analysis with repeated measures analysis (FIG. 9C) have been performed. The size of the donut represents the abundance of the metabolites, while the color of the donut represents the FC of ³C₆-glucose in the metabolite. For the additional metabolites, see FIG. 10. Abbreviations: FC=fractional contribution; negat siRNA=negative/non-targeting siRNA; AKR1B1 siRNA−=siRNA targeting ARK1B1 gene; CTR=control; P=patient; g=p-value reflecting effect of genotype; s=p-value reflecting the effect of the siRNA targeting AKR1B1; i=p-value reflecting the interaction between AKR1B1 KD and positional labeling.

FIGS. 10A-10C. FIG. 10A) Confirmation of AKR1B1 knock down by siRNA. AKR1B1 RT-qPCR (P2, P5 PMM2-CDG, t=1; healthy control n=1, t=1) and AKR1B1 Western blot (WB) (P2 PMM2-CDG, healthy control) were performed confirming AR knock-down. FIG. 10B) siRNA inhibition of AR gene (AKR1B1) resulted in global metabolic changes. Both abundance (represented by the size of the donut) and fractional contribution of ¹³C₆ glucose (represented by the colored part of the donut) of several metabolites related to glycolysis, TCA, hexosamine biosynthesis, and sugar nucleotide synthesis significantly change upon AKR1B1 KD. PMM2-CDG n=2, t=2; healthy control n=2, t=1-2. FIG. 10C) Inhibition of aldose reductase had a positive effect on cell proliferation. Confluence change rate was calculated as the difference in the confluence between the first and last day of the experiment, divided by the number of days experiment lasted. Control n=3, t=1, PMM2-CDG n=2, t=1. Two-way repeated measures ANOVA was used for statistical analysis. Abbreviations: CTR=control; FC=fractional contribution; negat siRNA=negative/non-targeting siRNA; AKR1B1 siRNA=siRNA targeting ARK1B1 gene; P=phosphate; *=p-value reflecting effect of ARK1B1 siRNA on abundance; #=p-value effect of AKR1B1 siRNA on FC; P #=patient.

### DETAILED DESCRIPTION

This document provides methods and materials for treating a mammal (e.g., a human such as a juvenile human) having one or more CDG (e.g., PMM2-CDG). For example, (a) one or more AR inhibitors (e.g., epalrestat) and (b) one or more nucleosides (e.g., guanosine, uridine, adenosine, thymidine, and/or cytidine) can be administered to a mammal having one or more CDG (e.g., PMM2-CDG) to treat the mammal.

In some cases, (a) one or more (e.g., one, two, three, four, or more) AR inhibitors (e.g., epalrestat) and (b) one or more (e.g., one, two, three, four, or more) nucleosides (e.g., guanosine, uridine, adenosine, thymidine, and/or cytidine) can be administered to a mammal (e.g., a human such as a juvenile human) having one or more CDG such as PMM2-CDG) to reduce the severity of one or more CDG (e.g., PMM2-CDG). For example, (a) one or more (e.g., one, two, three, four, or more) AR inhibitors (e.g., epalrestat) and (b) one or more (e.g., one, two, three, four, or more) nucleosides (e.g., guanosine, uridine, adenosine, thymidine, and/or cytidine) can be administered to a mammal (e.g., a human such as a juvenile human) in need thereof (e.g., a human such as a juvenile human having one or more CDG such as PMM2-CDG) to reduce or eliminate one or more symptoms of a CDG (e.g., PMM2-CDG) within the mammal. Examples of symptoms that can be reduced or eliminated in a mammal having one or more CDG (e.g., PMM2-CDG) as described herein (e.g., by administering one or more AR inhibitors (e.g., epalrestat) and one or more nucleosides (e.g., guanosine, uridine, adenosine, thymidine, and/or cytidine)) include, without limitation, ataxia, speech delay, seizures, global developmental delay, intellectual disability, coagulation abnormalities, and liver abnormalities. For example, (a) one or more AR inhibitors (e.g., epalrestat) and (b) one or more nucleosides (e.g., guanosine, uridine, adenosine, thymidine, and/or cytidine) can be administered to a mammal having one or more CDG (e.g., PMM2-CDG) to reduce one or more symptoms the CDG within the mammal by, for example, 10, 20, 30, 40, 50, 60, 70, 80, 90, 95, or more percent. For example, (a) one or more AR inhibitors (e.g., epalrestat) and (b) one or more nucleosides (e.g., guanosine, uridine, adenosine, thymidine, and/or cytidine) can be administered to a mammal having one or more CDG (e.g., PMM2-CDG) to reduce one or more symptoms the CDG within the mammal by, for example, at least 1.1 fold (e.g., about 1.1 fold, about 1.5 fold, about 2 fold, about 2.5 fold, about 3 fold, about 3.5 fold, about 4 fold, about 5 fold, about 6 fold, or more).

In some cases, (a) one or more AR inhibitors (e.g., epalrestat) and (b) one or more nucleosides (e.g., guanosine, uridine, adenosine, thymidine, and/or cytidine) can be administered to a mammal (e.g., a human such as a juvenile human) in need thereof (e.g., a human such as a juvenile human having one or more CDG such as PMM2-CDG) to increase a level of one or more PMM2-related metabolites (e.g., one or more metabolites associated with PMM2 such as a metabolite in a nucleotide and/or sugar nucleotide biosynthesis pathway associated with PMM2) within the mammal. Examples of PMM2-related metabolites that can be increased in a mammal having one or more CDG (e.g., PMM2-CDG) as described herein (e.g., by administering (a) one or more AR inhibitors (e.g., epalrestat) and (b) one or more nucleosides (e.g., guanosine, uridine, adenosine, thymidine, and/or cytidine)) include, without limitation, hexose-phosphates (e.g. man-1-P, mannose-6-phosphate (man-6-P)), hexose-bisphosphates (e.g. glucose-1,6-diphosphate (Glc-1,6-P2)), nucleosides (e.g. guanosine, uridine, adenosine, thymidine, and cytidine), nucleotides (e.g. guanosine triphosphate (GTP), guanosine diphosphate (GDP)), and nucleotide sugars (e.g. GDP-mannose, GDP-fucose, UDP-hexose, UDP-HexNAc). For example, (a) one or more AR inhibitors (e.g., epalrestat) and (b) one or more nucleosides (e.g., guanosine, uridine, adenosine, thymidine, and/or cytidine) can be administered to a mammal having one or more CDG (e.g., PMM2-CDG) to increase the level of one or more PMM2-related metabolites within the mammal by, for example, 5, 10, 20, 30, 40, 50, 60, 70, 80, 90, 95, or more percent. For example, (a) one or more AR inhibitors (e.g., epalrestat) and (b) one or more nucleosides (e.g., guanosine, uridine, adenosine, thymidine, and/or cytidine) can be administered to a mammal having one or more CDG (e.g., PMM2-CDG) to increase the level of one or more PMM2-related metabolites within the mammal by, for example, at least 1.5 fold (e.g., about 1.5 fold, about 2 fold, about 2.5 fold, about 3 fold, about 3.5 fold, about 4 fold, about 5 fold, about 6 fold, or more).

Any appropriate mammal having one or more CDG (e.g., PMM2-CDG) can be treated as described herein (e.g., by administering (a) one or more AR inhibitors (e.g., epalrestat) and (b) one or more nucleosides (e.g., guanosine, uridine, adenosine, thymidine, and/or cytidine)). Examples of mammals that can be treated as described herein include, without limitation, humans, non-human primates (e.g., monkeys), horses, bovine species, porcine species, dogs, cats, mice, rats, rabbits, and goats. In some cases, an animal that is not a mammal can be treated as described herein (e.g., by administering (a) one or more AR inhibitors (e.g., epalrestat) and (b) one or more nucleosides (e.g., guanosine, uridine, adenosine, thymidine, and/or cytidine)). For example, a fish (e.g., a zebrafish) can be treated as described herein. In some cases, a human (e.g., a human having one or more CDG (e.g., PMM2-CDG)) can be treated as described herein. In some cases, a juvenile mammal (e.g., a juvenile mammal having one or more CDG such as PMM2-CDG) can be treated as described herein. For example, a juvenile human can be administered (a) one or more AR inhibitors (e.g., epalrestat) and (b) one or more nucleosides (e.g., guanosine, uridine, adenosine, thymidine, and/or cytidine) as described herein. For example, a human that is from about 1 month of age to about 17 years of age can be administered (a) one or more AR inhibitors (e.g., epalrestat) and (b) one or more nucleosides (e.g., guanosine, uridine, adenosine, thymidine, and/or cytidine) as described herein. In some cases, an adult mammal (e.g., an adult mammal having one or more CDG such as PMM2-CDG) can be treated as described herein. For example, an adult human can be administered (a) one or more AR inhibitors (e.g., epalrestat) and (b) one or more nucleosides (e.g., guanosine, uridine, adenosine, thymidine, and/or cytidine) as described herein. For example, a human that is at least 18 years of age can be administered (a) one or more AR inhibitors (e.g., epalrestat) and (b) one or more nucleosides (e.g., guanosine, uridine, adenosine, thymidine, and/or cytidine) as described herein.

A mammal (e.g., a human such as a juvenile human) being treated as described herein (e.g., by administering (a) one or more AR inhibitors (e.g., epalrestat) and (b) one or more nucleosides (e.g., guanosine, uridine, adenosine, thymidine, and/or cytidine)) can have any one or more types of CDG Examples of CDG that can be treated as described herein include, without limitation, PMM2-CDG, GMMPA-CDG, GMMPB deficiency, DPM1-CDG, DPM2-CDG, DPM3-CDG, ALG1-CDG, ALG2-CDG, ALG3-CDG, ALG6-CDG, ALG8-CDG, ALG9-CDG, ALG11-CDG, ALG12-CDG, MAN1B1-CDG, MAN2B2-CDG, FCSK-CDG, GFUS-CDG, SLC35C1-CDG, and FUT8-CDG.

In some cases, the methods described herein can include identifying a mammal (e.g., a human such as a juvenile human) as having one or more CDG (e.g., PMM2-CDG). Any appropriate method can be used to identify a mammal as having one or more CDG (e.g., PMM2-CDG). Examples of methods for identifying a mammal as having one or more CDG (e.g., PMM2-CDG) include, without limitation, genetic analysis, enzyme assays for a specific enzyme coded by the affected gene (e.g. a PMM2 enzymatic activity assay), detection of elevated urine polyol (e.g., sorbitol and mannitol) levels, detection of abnormal glycosylation (e.g. methods based on carbohydrate deficient transferrin analysis), detection of abnormal coagulation (ATIII-antithrombin III), impaired liver function (e.g., altered levels of ALT and/or AST), and detection of neurologic symptoms (e.g. ataxia).

A mammal (e.g., a human such as a juvenile human) having one or more CDG (e.g., PMM2-CDG) can be administered or instructed to self-administer any appropriate one or more (e.g., one, two, three, four, or more) AR inhibitors as described herein. An AR inhibitor can inhibit AR polypeptide activity or can inhibit AR polypeptide expression. Examples of compounds that can inhibit AR polypeptide activity include, without limitation, antibodies (e.g., neutralizing antibodies) that target (e.g., target and bind) to an AR polypeptide, and small molecules (e.g., competitive inhibitors) that target (e.g., target and bind) to an AR polypeptide. Examples of compounds that can inhibit of AR polypeptide expression include, without limitation, nucleic acid molecules designed to induce RNA interference of polypeptide expression of an AR polypeptide (e.g., a siRNA molecule or a shRNA molecule), antisense molecules, miRNAs, and nucleic acid molecules designed to induce CRISPR interference (CRISPRi). Examples of AR inhibitors that can be administered to mammal (e.g., a human such as a juvenile human) having one or more CDG (e.g., PMM2-CDG) as described herein include, without limitation, epalrestat, AT007, fidarestat, sorbinil, and salfredin b11.

A mammal (e.g., a human such as a juvenile human) having one or more CDG (e.g., PMM2-CDG) can be administered or instructed to self-administer any appropriate one or more (e.g., one, two, three, four, or more) nucleosides as described herein. Examples of nucleosides that can be administered to mammal (e.g., a human such as a juvenile human) having one or more CDG (e.g., PMM2-CDG) as described herein include, without limitation, guanosine, uridine, adenosine, thymidine, and cytidine.

In some cases, in addition to or as an alternative to one or more (e.g., one, two, three, four, or more) nucleosides, a mammal (e.g., a human such as a juvenile human) having one or more CDG (e.g., PMM2-CDG) can be administered or instructed to self-administer any appropriate one or more (e.g., one, two, three, four, or more) nucleotides. For example, a nucleotide that can be administered to a mammal (e.g., a human such as a juvenile human) having one or more CDG (e.g., PMM2-CDG) can be a nucleotide monophosphate (e.g., can have one phosphate group). For example, a nucleotide that can be administered to a mammal (e.g., a human such as a juvenile human) having one or more CDG (e.g., PMM2-CDG) can be a nucleotide diphosphate (e.g., can have two phosphate groups). For example, a nucleotide that can be administered to a mammal (e.g., a human such as a juvenile human) having one or more CDG (e.g., PMM2-CDG) can be a nucleotide triphosphate (e.g., can have three phosphate groups). Examples of nucleotides that can be administered to mammal (e.g., a human such as a juvenile human) having one or more CDGs (e.g., PMM2-CDG) as described herein include, without limitation, guanosine monophosphate, guanosine diphosphate, guanosine triphosphate, uridine monophosphate, uridine diphosphate, uridine triphosphate, adenosine monophosphate, adenosine diphosphate, adenosine triphosphate, thymidine monophosphate, thymidine diphosphate, thymidine triphosphate, cytidine monophosphate, cytidine diphosphate, and cytidine triphosphate.

In some cases, (a) one or more AR inhibitors (e.g., epalrestat) and (b) one or more nucleosides (e.g., guanosine, uridine, adenosine, thymidine, and/or cytidine) can be formulated into a composition (e.g., a pharmaceutically acceptable composition) for administration to a mammal (e.g., a human such as a juvenile human) having one or more CDG (e.g., PMM2-CDG). For example, one or more AR inhibitors (e.g., epalrestat) and one or more nucleosides (e.g., guanosine, uridine, adenosine, thymidine, and/or cytidine) can be formulated into a single composition. In some cases, (a) one or more AR inhibitors (e.g., epalrestat) and (b) one or more nucleosides (e.g., guanosine, uridine, adenosine, thymidine, and/or cytidine) can be formulated together with one or more pharmaceutically acceptable carriers (additives), excipients, and/or diluents. In some cases, a pharmaceutically acceptable carrier, excipient, or diluent can be a naturally occurring pharmaceutically acceptable carrier, excipient, or diluent. In some cases, a pharmaceutically acceptable carrier, excipient, or diluent can be a non-naturally occurring (e.g., an artificial or synthetic) pharmaceutically acceptable carrier, excipient, or diluent. Examples of pharmaceutically acceptable carriers, excipients, and diluents that can be used in a composition described herein (e.g., a pharmaceutically acceptable composition including (a) one or more AR inhibitors (e.g., epalrestat) and (b) one or more nucleosides (e.g., guanosine, uridine, adenosine, thymidine, and/or cytidine)) include, without limitation, serum proteins (e.g., human serum albumin), water, and salts or electrolytes (e.g., phosphate salts, saline, and protamine sulfate).

In some cases, the one or more AR inhibitors and the one or more nucleosides can be administered to the mammal at the same time. For example, (a) one or more AR inhibitors (e.g., epalrestat) and (b) one or more nucleosides (e.g., guanosine, uridine, adenosine, thymidine, and/or cytidine) can be administered to a mammal (e.g., a human such as a juvenile human) having one or more CDG (e.g., PMM2-CDG) in a single composition containing both the one or more AR inhibitors and the one or more nucleosides.

In some cases, the one or more AR inhibitors and the one or more nucleosides can be administered to the mammal separately.

One or more AR inhibitors (e.g., epalrestat) and one or more nucleosides (e.g., guanosine, uridine, adenosine, thymidine, and/or cytidine) can be administered to a mammal (e.g., a human such as a juvenile human) by any appropriate route (e.g., oral, intranasal, inhalation, transdermal, and parenteral). One or more AR inhibitors (e.g., epalrestat) and one or more nucleosides (e.g., guanosine, uridine, adenosine, thymidine, and/or cytidine) can be administered to a mammal locally or systemically. For example, (a) one or more AR inhibitors (e.g., epalrestat) and (b) one or more nucleosides (e.g., guanosine, uridine, adenosine, thymidine, and/or cytidine) can be administered systemically by oral administration to a mammal (e.g., a human such as a juvenile human). When a mammal (e.g., a human such as a juvenile human) is administered (a) one or more AR inhibitors (e.g., epalrestat) and (b) one or more nucleosides (e.g., guanosine, uridine, adenosine, thymidine, and/or cytidine), and the one or more AR inhibitors and the one or more nucleosides (e.g., guanosine) are administered separately, the one or more AR inhibitors the one or more nucleosides can be administered by the same route or by different routes.

One or more AR inhibitors (e.g., epalrestat) can be administered to a mammal (e.g., a human such as a juvenile human) having one or more CDG (e.g., PMM2-CDG) in any appropriate amount (e.g., any appropriate dose). An effective amount of one or more AR inhibitors (e.g., epalrestat) can be any amount that increases a level of PMM2-related metabolites within the mammal being treated without producing significant toxicity to the mammal. In cases where one or more AR inhibitors includes epalrestat, an effective amount of epalrestat can be from about 0.5 milligrams of epalrestat per kilogram body weight (mg/kg) per day to about 150 mg/kg per day (e.g., from about 0.5 mg/kg per day to about 120 mg/kg per day, from about 0.5 mg/kg per day to about 100 mg/kg per day, from about 0.5 mg/kg per day to about 75 mg/kg per day, from about 0.5 mg/kg per day to about 50 mg/kg per day, from about 0.5 mg/kg per day to about 30 mg/kg per day, from about 0.5 mg/kg per day to about 25 mg/kg per day, from about 0.5 mg/kg per day to about 20 mg/kg per day, from about 0.5 mg/kg per day to about 15 mg/kg per day, from about 0.5 mg/kg per day to about 10 mg/kg per day, from about 0.5 mg/kg per day to about 5 mg/kg per day, from about 0.5 mg/kg per day to about 2 mg/kg per day, from about 5 mg/kg per day to about 150 mg/kg per day, from about 10 mg/kg per day to about 150 mg/kg per day, from about 25 mg/kg per day to about 150 mg/kg per day, from about 50 mg/kg per day to about 150 mg/kg per day, from about 75 mg/kg per day to about 150 mg/kg per day, from about 100 mg/kg per day to about 150 mg/kg per day, from about 125 mg/kg per day to about 150 mg/kg per day, from about 1 mg/kg per day to about 125 mg/kg per day, from about 2 mg/kg per day to about 100 mg/kg per day, from about 5 mg/kg per day to about 75 mg/kg per day, from about 10 mg/kg per day to about 50 mg/kg per day, from about 15 mg/kg per day to about 25 mg/kg per day, from about 1 mg/kg per day to about 25 mg/kg per day, from about 25 mg/kg per day to about 50 mg/kg per day, from about 50 mg/kg per day to about 75 mg/kg per day, from about 75 mg/kg per day to about 100 mg/kg per day, or from about 100 mg/kg per day to about 125 mg/kg per day). In cases where a mammal (e.g., a human such as a juvenile human) is also administered one or more nucleosides (e.g., guanosine), and the one or more nucleosides includes guanosine, an effective amount of guanosine can be from about 60 mg/kg per day to about 120 mg/kg per day (e.g., from about 60 mg/kg per day to about 100 mg/kg per day, from about 60 mg/kg per day to about 90 mg/kg per day, from about 60 mg/kg per day to about 80 mg/kg per day, from about 60 mg/kg per day to about 70 mg/kg per day, from about 70 mg/kg per day to about 120 mg/kg per day, from about 80 mg/kg per day to about 120 mg/kg per day, from about 90 mg/kg per day to about 120 mg/kg per day, from about 1000 mg/kg per day to about 120 mg/kg per day, from about 70 mg/kg per day to about 110 mg/kg per day, from about 80 mg/kg per day to about 100 mg/kg per day, from about 70 mg/kg per day to about 80 mg/kg per day, from about 80 mg/kg per day to about 90 mg/kg per day, from about 90 mg/kg per day to about 100 mg/kg per day, from about 100 mg/kg per day to about 110 mg/kg per day). The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the one or more CDG (e.g., PMM2-CDG) in the mammal being treated may require an increase or decrease in the actual effective amount administered.

One or more AR inhibitors (e.g., epalrestat) and one or more nucleosides (e.g., guanosine, uridine, adenosine, thymidine, and/or cytidine) can be administered to a mammal (e.g., a human such as a juvenile human) having one or more CDG (e.g., PMM2-CDG) at any appropriate frequency. The frequency of administration can be any frequency that increases a level of PMM2-related metabolites within the mammal being treated without producing significant toxicity to the mammal. For example, the frequency of administration can be daily. When a mammal (e.g., a human such as a juvenile human) is administered (a) one or more AR inhibitors (e.g., epalrestat) and (b) one or more nucleosides (e.g., guanosine, uridine, adenosine, thymidine, and/or cytidine) separately, the one or more AR inhibitors and the one or more nucleosides can be administered at the same frequency or at different frequencies. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

One or more AR inhibitors (e.g., epalrestat) and one or more nucleosides (e.g., guanosine, uridine, adenosine, thymidine, and/or cytidine) can be administered to a mammal (e.g., a human such as a juvenile human) having one or more CDG (e.g., PMM2-CDG) for any appropriate duration. An effective duration for administering or using (a) one or more AR inhibitors (e.g., epalrestat) and (b) one or more nucleosides (e.g., guanosine, uridine, adenosine, thymidine, and/or cytidine) can be any duration that increases a level of PMM2-related metabolites within the mammal being treated without producing significant toxicity to the mammal. For example, the effective duration can vary from several months to several years or from several years to a lifetime. Multiple factors can influence the actual effective duration used for a particular treatment. For example, an effective duration can vary with the frequency of administration, effective amount, use of multiple treatment agents, and/or route of administration.

In some cases, methods for treating a mammal (e.g., a human such as a juvenile human) as described herein (e.g., by administering (a) one or more AR inhibitors (e.g., epalrestat) and (b) one or more nucleosides (e.g., guanosine, uridine, adenosine, thymidine, and/or cytidine)) can include administering to the mammal (a) one or more AR inhibitors (e.g., epalrestat) and (b) one or more nucleosides (e.g., guanosine, uridine, adenosine, thymidine, and/or cytidine) as the sole active ingredient(s). For example, a composition containing (a) one or more AR inhibitors (e.g., epalrestat) and (b) one or more nucleosides (e.g., guanosine, uridine, adenosine, thymidine, and/or cytidine) can include the one or more AR inhibitors and the one or more nucleosides as the sole active ingredient(s) in the composition for treating a mammal (e.g., a human such as a juvenile human) having one or more CDG (e.g., PMM2-CDG).

In some cases, methods for treating a mammal (e.g., a human such as a juvenile human) as described herein (e.g., by administering (a) one or more AR inhibitors (e.g., epalrestat) and (b) one or more nucleosides (e.g., guanosine, uridine, adenosine, thymidine, and/or cytidine)) also can include administering to the mammal one or more (e.g., one, two, three, or more) additional agents used to treat a CDG and/or performing one or more (e.g., one, two, three, or more) therapies used to treat a CDG For example, a combination therapy used to treat a mammal (e.g., a human such as a juvenile human) having one or more CDG (e.g., PMM2-CDG) can include administering to the mammal (a) one or more AR inhibitors (e.g., epalrestat) and (b) one or more nucleosides (e.g., guanosine, uridine, adenosine, thymidine, and/or cytidine), and administering to the mammal one or more (e.g., one, two, three, or more) additional agents used to treat a CDG (e.g., additional agents used to manage one or more symptoms of a CDG (e.g., PMM2-CDG)). In cases where (a) one or more AR inhibitors (e.g., epalrestat) and (b) one or more nucleosides (e.g., guanosine, uridine, adenosine, thymidine, and/or cytidine) are used in combination with additional agents used to treat a mammal (e.g., a human such as a juvenile human) having one or more CDG (e.g., PM2-CDG), the one or more additional agents can be administered at the same time (e.g., in a single composition containing (a) one or more AR inhibitors (e.g., epalrestat) and (b) one or more nucleosides (e.g., guanosine, uridine, adenosine, thymidine, and/or cytidine), and containing the one or more additional agents) or independently. For example, a composition including (a) one or more AR inhibitors (e.g., epalrestat) and (b) one or more nucleosides (e.g., guanosine, uridine, adenosine, thymidine, and/or cytidine) can be administered first, and the one or more additional agents administered second, or vice versa.

In some cases, a combination therapy used to treat a mammal (e.g., a human such as a juvenile human) having one or more CDG (e.g., PMM2-CDG) can include administering to the mammal (a) one or more AR inhibitors (e.g., epalrestat) and (b) one or more nucleosides (e.g., guanosine, uridine, adenosine, thymidine, and/or cytidine), and can include performing one or more (e.g., one, two, three, or more) therapies used to treat a CDG Examples of therapies that can be used to treat a mammal (e.g., a human such as a juvenile human) having one or more CDG (e.g., PMM2-CDG) include, without limitation, dietary fructose restriction, and/or ketogenic diets. In cases where (a) one or more AR inhibitors (e.g., epalrestat) and (b) one or more nucleosides (e.g., guanosine, uridine, adenosine, thymidine, and/or cytidine) are used in combination with one or more therapies used to treat a mammal (e.g., a human such as a juvenile human) having one or more CDG (e.g., PMM2-CDG), the one or more additional therapies can be performed at the same time or independently of the administration of the one or more AR inhibitors and the one or more nucleosides. For example, (a) one or more AR inhibitors (e.g., epalrestat) and (b) one or more nucleosides (e.g., guanosine, uridine, adenosine, thymidine, and/or cytidine) can be administered before, during, or after the one or more additional therapies are performed.

In some cases, a course of treatment can be monitored as described herein. For example, the levels of PMM2-related metabolites within the mammal can be monitored over the course of treatment to determine whether or not the treatment is effective and/or remains effective over time.

The invention will be further described in the following examples, which do not limit the scope of the invention described in the claims.

### EXAMPLES

### Example 1: Epalrestat and PMM2-CDG

This Example identifies epalrestat as an AR inhibitor and describes using nucleoside supplementation together with epalrestat as a therapeutic for PMM2-CDG patients.

This Example also describes how administration of epalrestat, and optionally supplementation with nucleosides, can restore GDP-mannose levels.

### Methods

### Human Subjects

Fifty individuals with PMM2-CDG were included in this study (Table 1). Urine samples (P1-P50) and blood samples (P5-P7) were collected.

TABLE 1
Demographic, genetic, and biochemical characteristics of the patients screened
for urine polyols. Highest measured urine sorbitol (normal <5 mmol/mmol
creatine), mannitol (normal <20 mmol/mmol creatine). Mono-oligo/dioligo
ratio (normal <=0.06); A-oligo/dioligo ratio (normal <=0.011).

| | | | | Highest | Highest |
|---|---|---|---|---|---|
| | | | | measured urine | measured urine |
| | | | | Sorbitol <5 | Mannitol <20 |
| | | | Age#/ | mmol/mol | mmol/mol |
| ID | Gene | Nucleotide change | Sex | CT | CT |
| P1 | PMM2 | c.422G > A, c.722G > C | 26/F | 7 | 23 |
| P2*+ | PMM2 | c.422G > A, c.336C > T | 47/F | 41 | 69 |
| P3*+ | PMM2 | c.422G > A, c.336C > T | 47/F | 41 | 113 |
| P4* | PMM2 | c.422G > A, c.647A > T | 9/M | 5.67 | 121.74 |
| P5* | PMM2 | c.415G > A, c.422G > A | 9/F | 19.92 | [symbol] 4 [symbol] .6 |
| P6* | PMM2 | c.422G > A, c.548T > C | 8/M | 50.80 | 23.90 |
| P7 | PMM2 | c.422G > A, c.647A > T | 3/F | [symbol] . [symbol] | 10.37 |
| P8* | PMM2 | c.357C > A, c.422G > A | 9/M | 130.53 | 194.13 |
| P9* | PMM2 | c.422G > A, c.203T > G | 3/M | 12.76 | 9.01 |
| P10* | PMM2 | c.109C > T, c.337C > A | 4/M | 24.73 | 52.94 |
| P11* | PMM2 | c.96A > C, c.140C > T | 11/M | 3 | 6 |
| P12* | PMM2 | c.422G > A, c.722G > C | 14/M | 12.45 | 16.44 |
| P13* | PMM2 | c.422G > a, c.458T > C | 18/M | 3.99 | 3.64 |
| P14* | PMM2 | c.368G > A, c.722G > C | 25/F | 5.74 | 12.04 |
| P15* | PMM2 | c.647A > G, c.415G > A | 71/M | 2.80 | 7.99 |
| P16* | PMM2 | c.422G > A, c.640-23A > G | 13/F | 5.86 | 22. [symbol] |
| P17* | PMM2 | c.422 G > A, c.640-23A > G | 11/M | [symbol] . [symbol] | [symbol] . [symbol] |
| P18* | PMM2 | c.338C > T, c.422G > A | 10/M | 5.16 | 6.96 |
| P19* | PMM2 | c.563A > G, c.691G > A | 10/F | 14.7 | 59.2 |
| P20* | PMM2 | c.422 G > A, c.365G > A | 7/M | 14.80 | 28.46 |
| P21* | PMM2 | c.422G > A, c.357C > A | 5/M | 4 [symbol] .73 | 4 [symbol] .4 |
| P22* | PMM2 | c.385G > A, c.422G > A | 6/F | 14.65 | 12.55 |
| P23* | PMM2 | c.691G > A, c.422G > A | 6/M | 19.69 | 21.09 |
| P24* | PMM2 | c.422G > A, c.713G > A | 10/M | 7.47 | 33.52 |
| P25* | PMM2 | c.422G > A, c.623G > C | 12/F | 2.69 | 7.03 |
| P26 | PMM2 | c.422G > A, c.686A > C | 15/F | 14. [symbol] | 14.65 |
| P27 | PMM2 | c.422G > A, c.722G > C | 21/F | 5.65 | 8.48 |
| P28 | PMM2 | c.323C > T, c.710C > G | 15/M | 11.87 | 9.52 |
| P29 | PMM2 | c.422G > A, c.44G > C | 4/M | 12.79 | 13.65 |
| P30 | PMM2 | c.323C > T, c.710C > G | 11/M | 5.1 [symbol] | 8.66 |
| P31 | PMM2 | c.26G > A, c.442G > A | 26/M | 1.03 | 2.52 |
| P32 | PMM2 | c.710C > G, c.728T > C | 2/F | 13.12 | 5.57 |

[symbol]

indicates data missing or illegible when filed

### Methods

### Human Subjects

Fifty individuals with PM/2-CDG were included in this study (Table 1). Urine samples (P1-P50) and blood samples (P5-P7) were collected.

TABLE 1
Demographic, genetic, and biochemical characteristics of the patients screened
for urine polyols. Highest measured urine sorbitol (normal <5 mmol/mmol
creatine), mannitol (normal <20 mmol/mmol creatine). Mono-oligo/dioligo
ratio (normal <=0.06); A-oligo/dioligo ratio (normal <=0.011).

| | | | | Highest | Highest |
|---|---|---|---|---|---|
| | | | | measured urine | measured urine |
| | | | | Sorbitol <5 | Mannitol <20 |
| | | | Age#/ | mmol/mol | mmol/mol |
| ID | Gene | Nucleotide change | Sex | CT | CT |
| P1 | PMM2 | c.422G > A, c.722G > C | 26/F | 7 | 23 |
| P2*+ | PMM2 | c.422G > A, c.338C > T | 47/F | 41 | 69 |
| P3*+ | PMM2 | c.422G > A, c.338C > T | 47/F | 41 | 113 |
| P4* | PMM2 | c.422G > A, c.647A > T | 9/M | 5.67 | 121.74 |
| P5* | PMM2 | c.415G > A, c.422G > A | 9/F | 19.93 | 648.6 |
| P6* | PMM2 | c.422G > A, c.548T > C | 8/M | 50.80 | 23.90 |
| P7 | PMM2 | c.422G > A, c.647A > T | 3/F | 9.85 | 10.37 |
| P8* | PMM2 | c.357C > A, c.422G > A | 9/M | 130.53 | 194.13 |
| P9* | PMM2 | c.422G > A, c.203T > G | 3/M | 12.76 | 9.01 |
| P10* | PMM2 | c.109C > T, c.337C > A | 4/M | 24.23 | 52.94 |
| P11* | PMM2 | c.98A > C, c. 140C > T | 11/M | 3 | 6 |
| P12* | PMM2 | c.422G > A, c.722G > C | 14/M | 12.45 | 16.44 |
| P13* | PMM2 | c.422G > A, c.458T > C | 18/M | 3.99 | 3.64 |
| P14* | PMM2 | c.368G > A, c.722G > C | 25/F | 5.74 | 12.04 |
| P15* | PMM2 | c.647A > G, c.415G > A | 71/M | 2.80 | 7.99 |
| P16* | PMM2 | c.422 G > A, c.640-23A > G | 13/F | 5.86 | 22.55 |
| P17* | PMM2 | c.422 G > A, c.640-23A > G | 11/M | 30.09 | 89.87 |
| P18* | PMM2 | c.338C > T, c.422G > A | 10/M | 5.16 | 6.96 |
| P19* | PMM2 | c.563A > G, c.691G > A | 10/F | 14.7 | 59.2 |
| P20* | PMM2 | c.422 G > A, c.385G > A | 7/M | 14.80 | 28.46 |
| P21* | PMM2 | c.422G > A, c.357C > A | 5/M | 41.73 | 46.4 |
| P22* | PMM2 | c.385G > A, c.422G > A | 6/F | 14.65 | 12.55 |
| P23* | PMM2 | c.691G > A, c.422G > A | 6/M | 19.69 | 21.09 |
| P24* | PMM2 | c.422G > A, c.713G > A | 10/M | 7.47 | 33.52 |
| P25* | PMM2 | c.422G > A, c.623G > C | 12/F | 2.69 | 7.03 |
| P26 | PMM2 | c. 422G > A, c.686A > C | 15/F | 14.68 | 14.85 |
| P27 | PMM2 | c.422G > A, c.722G > C | 21/F | 5.65 | 8.48 |
| P28 | PMM2 | c.323C > T, c.710C > G | 15/M | 11.87 | 9.52 |
| P29 | PMM2 | c.422G > A, c.44G > C | 4/M | 12.79 | 13.65 |
| P30 | PMM2 | c.323C > T, c.710C > G | 11/M | 5.19 | 8.66 |
| P31 | PMM2 | c.26G > A, c.442G > A | 26/M | 1.03 | 2.52 |
| P32 | PMM2 | c.710C > G, c.728T > C | 2/F | 13.12 | 5.57 |
| P33 | PMM2 | c.422G > A, c.691G > A | 10/M | 8.49 | 138.29 |
| P34 | PMM2 | c.323C > T, c.422G > A | 4/M | 8.33 | 10.20 |
| P35 | PMM2 | c.548T > C | 5/M | 10.36 | 10.20 |
| P36 | PMM2 | c.422G > A, c.556G > A | 9/F | 11.46 | 19.66 |
| P37 | PMM2 | c.470T > C, c.710C > T | 15/M | 9.8 | 66.6 |
| P38 | PMM2 | c.422G > A, c.338C > T | 16/M | 5.13 | 10.19 |
| P39 | PMM2 | c.205 C > T, c.442G > A | 10/M | 5.36 | 8.68 |
| P40 | PMM2 | c.357C > A, c.639-1G > T | 27/M | 6.27 | 5.72 |
| P41 | PMM2 | c.442G > A, c.338C > T | 9/M | 13.25 | 47.51 |
| P42 | PMM2 | c.422G > A, c.337C > T | 10/M | 5.55 | 9.28 |
| P43 | PMM2 | c.458T > C, c.43G > A | 3/F | 7.13 | 30.32 |
| P44 | PMM2 | c.422G > A, c.647A > T | 5/F | 8.58 | 10.46 |
| P45 | PMM2 | c.722G > C, c.710 C > G | 8/F | 5.05 | 17.71 |
| P46\hat{ } | PMM2 | c.357C > A | 35/F | 8.44 | 39.63 |
| P47 | PMM2 | c.422G > A, c.707A > G | 4/F | 20.64 | 163.32 |
| P48 | PMM2 | c.422G > A, c.647A > T | 4/M | 17.82 | 160.78 |
| P49 | PMM2 | c.95_96delTAinsGC, c.422G > A | 11/F | 56.79 | 39.83 |
| P50 | PMM2 | c.422G > A, c.524-5G > A intronic | 3m/M | 14.62 | 17.94 |

Abnormal values are in bold. Abbreviations: ATIII = antithrombin III; M = male, F = female, P1-50 = patient 1-50; m = months.; # age at the time of the publication (years); & patient on Kepra that contains sorbitol, however, the amounts do not account for the increase seen in urine.
+patients first reported in (Jaeken et al., Clin. Chim. Acta, 144(2-3): 245-7 (1984));
*patients reported in (Ligezka et al, Ann. Neurol., 90(6): 887-900 (2021));
\hat{ }patient reported in (Vaes et al., JIMD Reports, 54: 16-21 (2020))

### Cell Culture

Patient (P1-P6) and control fibroblasts were obtained by a skin punch biopsy. The detailed clinical information of the six patients is given in Table 2. Fibroblasts were maintained in low glucose DMEM (Gibco) supplemented with 10% FBS in an incubator at 37 °C, 5% CO₂, no longer than two months or until they reached passage 15 to avoid senescence. Routine mycoplasma testing was performed.

### Zebrafish Strains, Maintenance, and Husbandry.

Animals were maintained according to standard protocols. The zebrafish strains were obtained from the Zebrafish International Resource Center (ZIRC, Eugene, OR). Pmm2sa10150 mutant zebrafish were established as described elsewhere (Klaver et al., JCI Insight, 6(24):e153474 (2021)).

### Outcomes

The primary objective of the study was to assess the efficacy and safety of epalrestat treatment in 3 PMM2-CDG pediatric subjects prior to a planned Phase III clinical trial (Oral epalrestat therapy in Pediatric Subjects with PMM2-CDG, NCT04925960). The secondary objective was to assess the ability of epalrestat to improve clinically relevant aspects of PMM2-CDG such as increased urine polyols (sorbitol, mannitol), abnormal glycosylation (carbohydrate deficient transferrin), abnormal coagulation (ATIII=antithrombin III), impaired liver function (ALT=Alanine Transaminase, AST=Aspartate Transaminase), neurologic function (Brief Ataxia Rating Scale=BARS), severity of phenotype (Nijmegen Pediatric CDG Rating Scale=NPCRS). The ability of epalrestat to inhibit polyol production was monitored by evaluating urine polyol levels (sorbitol, mannitol), which are elevated in PMM2-CDG. Next, carbohydrate deficient transferrin, which is abnormal in PMM2-CDG, was evaluated in order to assess improvement of glycosylation following epalrestat treatment. CDT mono-oligo/dioligo ratio and a-oligo/dioligo ratio are used to evaluate glycosylation abnormalities. Transferrin normally occurs with two glycan chains (di-oligo). In the case of glycosylation abnormalities, glycan chains are missing (A-oligo transferrin has no glycan chains, mono-oligo has one glycan chain, di-oligo has two glycan chains). The higher ratio of a-oligo/di-oligo and mono-oligo/di-oligo indicate glycosylation abnormalities are present. Therefore, improvement in a-oligo/di-oligo and mono-oligo/di-oligo ratios can be used to monitor improvement or worsening of glycosylation status. Changes in the liver enzymes AST, ALT, which are frequently elevated in PMM2-CDG, were monitored both as part of a safety study and as part of monitoring clinical improvement in coagulation. Neurologic improvement was assessed by modified BARS scores in 2 patients (3^{rd} patient was not eligible for the test due to age at the start of the trial). NPCRS was assessed at every visit to assess changes in the severity of the patients' phenotype. Additionally, the frequency of adverse events, changes in vital signs, complete blood count, and changes in BMI were monitored.

### Procedures

Two patients were started on epalrestat on single patient investigational new drug (IND) (P5 IRB: 19-010017; IND #145262; P6 Protocol PMM2-CDG-001/A). Epalrestat was initiated on the parents' own initiative in the third patient (P7). Two patients were in their second year of epalrestat treatment, while the third, was in the 3rd year of treatment. Physical, neurological, and biochemical examinations were performed in 3 PMM2-CDG patients at baseline, and at every three months after the start of the epalrestat treatment. Patients underwent NPCRS phenotype severity assessment and biological sampling at every visit. Epalrestat (Ono Pharmaceuticals, Osaka, Japan) was taken orally 3 times per day (TID) before meals in a divided dose, with dosage escalation throughout the treatment. The pharmacological profile of epalrestat was as described elsewhere (Ligezka et al, Ann. Neurol., 90(6):887-900 (2021)). The starting dosage in P5 and P6 was 0.8 mg/kg/day, while P7 was started on 0.5 mg/kg/day due to her young age. P7 dosage was increased to 0.8 mg/day after a month, once safety was established. The complete overview of the dosage escalation is given in Table 3. Blood and urine samples were collected routinely. Urine polyols, carbohydrate deficient transferrin (CDT), anti-thrombin III (ATIII), liver enzymes (ALT, AST), and complete blood count (CBC) were performed. Modified Brief Ataxia Rating Scale was used to assess ataxia in the 2 patients (P5, P6; P7 was not eligible for the test due to age). The modified BARS scale included Archimedes spiral test, but not the abnormalities of the ocular pursuit system and the scale range was 0-54. Goal attainment scale (GAS) was administered as described per study protocol (ClinicalTrials.gov Identifier: NCT04199000). Quantification of goal attainment levels is done on a 5-point scale ranging from −2 to +2, with 0 indicating the expected level of goal attainment. Three individual goals were established by each patient (parent) at baseline and the progress was annually assessed. Goals included: walking without assistance (Goal #1 P5, P6), communicating with strangers (Goal #2 P5, P6), independent toilet training (Goal #3 P5), to be able to have a healthy and independent life (Goal #3 P6), to be able to equally play with peers (Goal #1 P7), normalization of laboratory results (liver enzymes) (Goal #2 P7), and improved balance to stand and walk (Goal #3 P7).

### Fructose Tolerance Lest

Fructose tolerance test was performed in P5 according to the standard clinical protocol at fasting state with 1 g/kg oral fructose, and the first voided urine was collected.

### Human Sample Analysis

Urine from 50 PMM2-CDG individuals was analyzed for the presence of polyols (sorbitol, mannitol, etc.). Control reference range was established from n=533 individuals out of which male=226; female=267; pediatric (<18 years old)=324; adult (>18 year old). Collected blood samples were used for CDT analysis, ApoCIII analysis, CBC, liver enzymes, and coagulation factor analysis.

### PMM Enzymatic Activity Assay

PMM enzymatic activity assay was previously performed in patient fibroblasts by CLIA certified PMM enzymatic activity assay (Ligezka et al, Ann. Neurol., 90(6):887-900 (2021)). PMM enzymatic activity in the zebrafish was assessed as follows: 10 embryos per sample were homogenized by sonication in 200 μL enzymatic buffer (25 mmol/L HEPES (Sigma), 25 mmol/L KCl (Fisher), pH 7.1; 0.02% (w/v) Na-azide (Sigma)) and stored at −80 °C. PMM enzymatic activity was then measured.

### Metabolomic Profiling of Fibroblasts

Briefly, 15,000 fibroblasts were plated in 1.5 mL DMEM (Gibco, 5.5 mM glucose, 2 mM glutamine, 10% FBS) per well in two 6-well plates. The medium change was performed on day 2 and day 4. On day 6, the medium was removed, cells were washed with ice-cold 0.9% NaCl (Sigma) (physiological solution) and 250 μL of extraction buffer (80% MeOH, IS) was added to the cells. Next, cells were scraped and the cell extracts transferred to 1.5 mL Eppendorf tubes and placed overnight at −80 °C. Next, samples were centrifuged at 15,000 rpm, 4 °C, 20 minutes. The supernatant was transferred to a vial for direct analysis by LC/MS, while 100 μL of 200 mM NaOH was added to the (protein) pellet. Pellets were incubated at 95 °C for 30 minutes after which they were centrifuged at 5,000 rpm, 4 °C, 10 minutes. Supernatant was used to determine protein concentration by BCA protein assay kit (Pierce).

### Metabolomic Profiling of Zebrafish

10 embryos per sample were homogenized by sonication in extraction buffer (80% MeOH, IS). The metabolites were then precipitated at −80 °C overnight, after which the samples were centrifuged at 15,000 rpm, 20 minutes, 4 °C. Next, the supernatant was transferred to a fresh Eppendorf tube. The samples were centrifuged again at 15,000 rpm, 20 minutes, 4 °C to remove any leftover impurities and transferred to a fresh MS vial. Finally, the samples were analyzed by LC/MS.

### Epalrestat Experiments

Fibroblasts were plated in 6-well plates for LC/MS and T75 flasks for GC/MS analysis as described above. After 24 hours, medium was removed and cells washed with PBS. Then, DMEM containing 5.5 mM glucose, 2 mM glutamine, and 10% dialyzed FBS was added. Epalrestat was freshly prepared and fibroblasts were supplemented with 10 μM epalrestat daily. After 48 hours media was changed, and 48 hours after that, metabolites were extracted and measured by both LC/MS and GC/MS. Zebrafish embryos were genotyped at 2 days post fertilization (dpf), and were treated by adding the epalrestat (solubilized in DMSO) directly into the embryo media at 5 dpf. Control embryos were similarly treated with DMSO alone. Embryos were harvested for metabolic analyses 24 hours posttreatment. The final concentration of DMSO was equivalent between control and treatment, never exceeding 0.5%.

### Nucleoside Supplementation Experiments

Cells were plated in 6-well plates. 24 hours after plating, medium was changed to either A) DMEM medium containing 5.5 mM glucose, 2 mM glutamine, 10% dialyzed FBS supplemented with EmbryoMax nucleosides (100×) to the final concentration 0.03 mM per nucleotide (Sigma), or B) DMEM medium containing 5.5 mM glucose, 2 mM glutamine, and 10% dialyzed FBS. Epalrestat was given to the patient and control fibroblasts.

### Uridine Guanosine Supplementation Experiments

The experiments were performed as described above. Cells were plated in 6-well plates. 24 hours after plating medium was changed to either A) DMEM medium containing 0.03 mM guanosine or 0.03 mM uridine, 5.5 mM glucose, 2 mM glutamine, and 10% dialyzed FBS, or B) DMEM medium containing 5.5 mM glucose, 2 mM glutamine, and 10% dialyzed FBS. Epalrestat was given to the patient and control fibroblasts.

Inhibition of AKR1B1 by siRNA

Two representative patient cell lines (P2 most severe, P5 mild presentation) were selected for subsequent experiments. A titration of AKR1B1 siRNA (0 nM, 2.5 nM, 5 nM, 10 nM, 20 nM, and 40 nM) was performed in both patient and control fibroblasts. It was found that 5 nM siRNA concentration was the optimal dose, resulting in significant KD of the AKR1B1 and the minimal cell death.

For metabolite extraction, membrane bound sialic acid isolation and quantification, and RNA and protein extraction, 15,000 cells were plated in 6-well plates in 1.5 mL of DMEM (Gibco) containing 5.5 mM glucose, 2 mM glutamine, and 10% FBS. The following day, medium was removed and the cells were washed with 1 mL PBS before adding 1.2 mL of DMEM containing 5.5 mM glucose, 2 mM glutamine and 10% dialyzed serum. Then, 5 nM siRNA targeting AKR1B1 (Ambion, cat AM16704) and non-targeting (negative) siRNA (Ambion, cat AM4611) was prepared using Lipofectamine 2000 (Invitrogen) and OptiMEM (Gibco) according to the manufacturer's protocol (final volume of 300 μL). This siRNA mix was then added to the cells. After 48 hours, the medium was removed, and cells were washed with PBS, and DMEM medium containing 2 mM glutamine, 10% dialyzed FBS and either A) 5.5 mM ¹³C₆-glucose or B) 5.5 mM ¹²C₆-glucose was added. Cells were incubated for another 48 hours followed by membrane sialic acid isolation, and RNA, protein, and metabolite extraction. Extracted metabolites were subjected to LC/MS. RNA was used to generate cDNA and validate the AKR1B1 KD with RT-qPCR, while protein was used for Western Blotting.

### Membrane Sialic Acid Isolation and Quantification

Control and PMM2-CDG fibroblasts were plated in 6-well plates in DMEM containing 5.5 mM glucose, 2 mM glutamine, and 10% FBS, followed by inhibition of AKR1B1 by siRNA. Membranes were isolated. Briefly, medium was removed from the cells and the cells were washed 3 times with 1 mL Dulbecco PBS containing 0.901 mM CaCl₂ (Merck) and 0.492 mM MgCl₂ (Merck). Next, cells were incubated with 1 mg/100 mL EZ-Link-Sulfo-NHS-LC-Biotin (Thermo) in Dulbecco for 30 minutes, RT, shaking. Cells were then washed twice with 1 mL Dulbecco, non-reacted biotin was blocked with 1 mL 20 mM glycine in Dulbecco for 15 minutes, and cells washed again with 1 mL Dulbecco. Dulbecco was then removed, cells scraped in 200 μL RIPA buffer (with protease inhibitors) and transferred to a new Eppendorf tube. Samples were lysed on ice with 3 consecutive freeze-thaw cycles. Further, 30 μL dynabeads streptavidin T1 (Invitrogen) was added to each sample and the samples were washed twice with 500 μL 10 mM ammonium bicarbonate and neuraminidase buffer (100 mM Sodium Acetate Buffer with 2 mM CaCl₂ (Merck), pH 5.0). Neuraminidase buffer was removed and 500 μL PBS was added to the beads. 30 μL of prepared mix was added to each sample, and samples were incubated, shaking overnight at 4 °C. Samples were put on a dynabead rack (Invitrogen) and the supernatant was transferred to a new Eppendorf and used for protein concentration assay. Beads containing membrane fractions were washed 2 times with 500 μL lysis buffer (2% IGEPAL (Sigma), 1% Triton X-100 (Sigma), and 10% glycerol in PBS) and then washed with 1 mL PBS. Samples are centrifuged at 500 rcf, 5 minutes, 4 °C and PBS was removed. 100 μL neuraminidase buffer containing 0.05 U neuraminidase was added to each sample and samples were incubated overnight at 37 °C, shaking. Next, supernatant was transferred to a new Eppendorf tube and lyophilized at 4 °C. Finally, pellets were resuspended in 100 μL of extraction buffer (80% MeOH, IS). Sialic acid was measured by LC/MS as described below. El Maven Polly software was used to annotate sialic acid and determine fractional contribution of glucose in sialic acid.

### RNA Extraction

Fibroblasts incubated with negative siRNA or siRNA targeting AKR1B1 were used to determine gene expression of AKR1B1. Briefly, 500 μL of TRIZol (Fisher) was added to cells previously washed with PBS. The cells were scraped and transferred to a fresh 1.5 mL Eppendorf tube. After, the cells were kept overnight at −80 °C. Next, 100 μL of chloroform was added and the sample briefly shaken. The samples were then centrifuged for 25 minutes, 12000 rpm, 4 °C. The polar phase was transferred to a fresh Eppendorf tube and 250 μL of 70% cold ethanol was added. RNA was then isolated from the sample using the RNeasy® Mini kit (Qiagen). RNA concentration and purity were determined using a NanoDrop™ spectrophotometer (ThermoFisher).

cDNA Preparation

Superscript II kit (Invitrogen) was used to prepare cDNA from isolated RNA. Briefly, 0.5 g of RNA was pipetted into a 0.2 mL PCR tube and RNAse free water was added to the final volume of 10.5 μL. The sample was incubated with 2 μL of Random Primers (1/20 diluted) in a thermocycler for 10 minutes at 70 °C and then cooled to 4 °C. Master mix containing 4 μL first strand buffer (5× undiluted), 2 μL DTT, 1 μL dNTP and 0.5 μL SuperScript II was added to each sample. The samples were returned to the thermocycler and the following program was applied: 10 minutes 25 °C, 80 minutes 42 °C, 15 minutes 70 °C, and 4 °C indefinitely. After, the samples were stored at −80 °C before performing RT-qPCR.

Analysis of AKR1B1 Gene Expression by qRT-PCR

Primer mix was prepared for the gene of interest (AKR1B1) and housekeeping gene (BACT). 0.5 μL of Taqman primer (AKR1B1-hs01091533_g1; BACT hs99999903_m1), 0.5 μL TaqMan universal PCR mastermix buffer (2× undiluted) (Applied biosciences), and 2.5 μL of RNase-free water was added to 2 μL of previously prepared cDNA. The mixture was then pipetted to a 96-well PCR plate, and the plate was sealed and briefly centrifuged before being placed in a LightCycler® 96 real time PCR system (Roche). The following protocol was run: preincubation (60 seconds), amplification (95 °C, 30 seconds, 45 cycles), acquisition 60 °C, 30 seconds. The machine built-in protocol was used to perform melt curve analysis. Next, Ct values were exported from the program and analyzed. The 2 ct method was used to analyze the relative changes in gene expression normalized against house-keeping gene mRNA expression.

### Western Blotting

Cells were scraped in PBS, and centrifuged at 600 rpm, 4 °C, 10 minutes. PBS was removed and the cell pellet was stored at −80 °C overnight. RIPA buffer was added to the cell pellet and cells were lysed by 3 consecutive freeze-thaw cycles. Samples were centrifuged at 12,000 rpm, 4 °C, 10 minutes and the supernatant transferred to a new Eppendorf tube. Protein concentration was determined by BCA protein assay kit (Pierce). Total cell protein (20 μg) mixed with sample buffer (4× Bolt LDS sample buffer, 10% DTT) was denatured (30 minutes, 95 °C) and loaded onto a 10% Bolt Bis-Tris gel (ThermoFisher). The proteins were then separated by PAGE (200 V, 1.5 hour, RT). Next, protein transfer onto the nitrocellulose membrane (1620115 BioRad) was done using Bolt transfer buffer (Thermo Fisher) containing 10% methanol (MeOH) (3 h, 35V, 4 °C), followed by blocking of the membrane in Licor buffer (Oddysay) (1 hour, RT). Incubation with primary antibodies (mouse anti-AKR1B1 1:200, rabbit anti-ACTB 1:5000 Cat #AC026 ABclonal, 4 °C) in Intercept blocking buffer (Licor) was done overnight. Next, the membrane was washed 6×10 minutes with PBS-T (0.1% Tween® 20) at RT after which the membrane was incubated with secondary antibodies (donkey anti-mouse 1:5000 Invitrogen Cat #SA510172 DyLight 800, donkey anti-rabbit 1:5000 Invitrogen Cat #SA5-10042 DyLight 680) (1 hour, RT). Then, the membrane was again washed 6×10 minutes with PBS-T, 1×10 minutes PBS). Finally, Li-COR Oddysey Cx (700, 800-channel) was used to visualize the membrane and quantify AKR1B1 and ACTB bands.

### Proliferation Assay

Cells were plated in 96-well plates in triplicates using the adapted protocol from the metabolomics siRNA experiments. PKH26 red fluorescent cell linker mini kit for general cell membrane labeling (Sigma) was used according to the manufacturer's protocol. The next day, the medium was changed, and nontargeting (negative) and siRNA targeting AKR1B1 were added to the cells. The plate with the cells was then placed in the IncuCyte® live cell analysis system (Essen Bioscience) and the plate was read in 2 hour intervals for 5 days. Four pictures per well were taken. Medium was changed on day 3. Cell confluence was measured using built-in software. The confluence rate of change was calculated as the difference between the confluence on the first day after plating and the final day of experiment, divided by the number of days cells were in culture.

### Metabolite Measurement by LCMS

An ion-pairing liquid chromatography column (C18) was used to separate 10 μL of sample, after which the metabolites were resolved on a Thermo Fisher Q-Exactive Hybrid Quadrupole-Orbitrap Mass Spectrometer (negative ion mode). The ESI settings were as follows: 50 sheet gas flow rate, auxiliary gas flow rate 15, spray voltage of 4 kV, S-lens RF level of 60, and the capillary temperature at 350 °C. A full scan (resolution 140,000 at 200 m/z, AGC at 3 e6, 512 ms ion fill time, and 70-850 m/z scan range) was performed. The metabolites were identified according to their elution time and m/z ratios by using in-house metabolite standard library and El-Maven v0. 12.0/Polly™ Labeled LC-MS Workflow or Thermo Fisher Xcalibur software. The correction of naturally occurring carbon isotopes was also performed. Relative metabolite abundances were normalized to the internal standard and protein content.

### Sugar Quantification by GC/MS

For GC/MS measurements of hexose-P, hexose-bisphosphates, hexose and polyols, fibroblasts were cultured in a similar fashion as for LC/MS with the following adjustments: 1) an appropriate number of fibroblasts was plated in T75 flasks instead of 6-well plates to ensure the optimal amount of metabolite concentration necessary for GC/MS; 2) metabolites were extracted from cells with 2 mL 80% MeOH, IS and the supernatant was transferred to a fresh 2 mL Eppendorf tube. Standards containing metabolites of interest were prepared simultaneously and dried overnight together with cellular extracts by vacuum centrifugation at 4 °C. The following day, 20 μL of methoxyamine (MOX) (Sigma Aldrich) was added to the samples and they were incubated for 90 minutes at 37 °C. Next, 60 μL of N,O-bis(trimethylsilyl)trifluoroacetamide (TMS) (Sigma Aldrich) was added to the samples, which were then incubated at 60 °C for 30 minutes. Samples were kept overnight in a dry and cool place to allow further derivatization with TMS. The following day, samples were placed on GC/MS and metabolites measured by Agilent 7890A GC (Agilent Technologies) coupled with an HP-5 ms 5% phenyl methyl silox capillary column (30 m, 0.25 mm, 0.25 mm, Agilent Technologies), interfaced with a QQQ MS (Agilent 7000B, Agilent Technologies) and operating under ionization by electron impact at 70 eV. The temperature of injection port, interface, and ion source was 230 °C. The quadrupole temperature was maintained at 150 °C. 1 μL of sample was injected onto the machine at either 1:25 split ratio (polyol), 1:5 split ratio (hexoses) or in splitless mode (hexose-P and hexose-BIP). Constant helium flow was applied at 1 mL/minute. The temperature gradient applied was as follows: 2 minutes 100 °C, next 175 °C at Δ20 °C/minute, followed by an increase to 230 °C at Δ4 °C/minute, kept at 230 °C for 3 minutes, increased to 300 °C at Δ 40 °C/minute, and lastly kept at 300 °C for 5 minutes. The column was further baked for another 3 minutes at 325 °C after the gradient. Hexose-bisphosphates (SIM 387 m/z), hexoses (SIM 204, 205, 206, 207, 208), and polyols (319, 320, 321, 322, 333 m/z) standards were used to determine the elution time of each metabolite. Data was then analyzed with the Mass Hunter Workstation software with the Quantitative Analysis Version B.06.00/Build 6.0.388.0. Relative abundance of metabolites was normalized to protein content.

### Quantification of NADPH and NADP

Fibroblasts from three patient fibroblasts (P2, P4, P5) and two healthy controls were plated on day 1. The next day medium was changed. The cells were grown in the presence and absence of 10 M epalrestat for another 24 hours. The medium was then removed, cells were washed with cold saline solution (0.9% NaCl) and 250 μL of extraction buffer (50% MeOH, 30% acetonitrile, 20% H2O Tris-HCl pH 7.6) was added to the cells. The metabolites were precipitated overnight at −80 °C, samples were centrifugated at 20,000 rpm, 20 minutes, 4 °C, and supernatant was transferred to a fresh vial. Pellets were used for determination of protein concentration. Supernatant containing metabolites was analyzed by LC/MS less than 24 hours after the initial collection to avoid degradation of NADPH into NADP. Relative abundance of NADP and NADPH was obtained. NADP/NADPH ratio was determined by dividing abundances of NADP with the abundances of NADPH in the same sample.

### Measurement of Sorbitol and Mannitol in FBS

Briefly, 10 μL of dialyzed and non-dialyzed FBS was added to 990 μL of extraction buffer (80% MeOH, IS). The samples were further precipitated overnight at −80 °C. Samples were centrifuged and supernatant transferred to a fresh Eppendorf tube. Next, samples were dried by vacuum centrifugation at 4 °C. Derivatization with MOX and TMS as well as measurements of sorbitol and mannitol in FBS were done.

Zebrabox Motility Assay Progeny of a pmm2^{sa1050/+} (pmm2^{m/+}) mating were collected and raised in the light following standard protocols. 5 dpf embryos were placed one per well in a 12-well culture dish and treated daily with either 20 μM epalrestat in DMSO or DMSO only, refreshing the embryo media daily. Embryos were also fed a small number of rotifers daily once behavioral readings were completed. Locomotor activity (swim paths, speeds) was monitored for ten-minute intervals from 6-10 dpf using the Zebrabox System (ViewPoint Inc., Toronto, Canada). Data was analyzed and graphed using GraphPad Prism.

### Statistical Analysis

All statistical analysis has been performed using GraphPad prism for MacBook. Significant differences between patients and controls were calculated by t-test with Welch correction. Significant differences before and after treatment with epalrestat/siRNA inhibition of AKR1B1 were determined by using Two-way repeated measures ANOVA. Metaboanalyst was used to generate heatmaps. Fish data was analyzed with Two-way ANOVA and multiple comparisons with Sidak correction. Mixed model analysis with repeated measures and multiple comparisons with Sidak correction was performed on CMP-sialic acid positional labeling and zebrabox motility assay data. P-value cut off was 0.05.

### Results

### Disturbed Polyol Metabolism is a Hallmark of PMM2-CDG

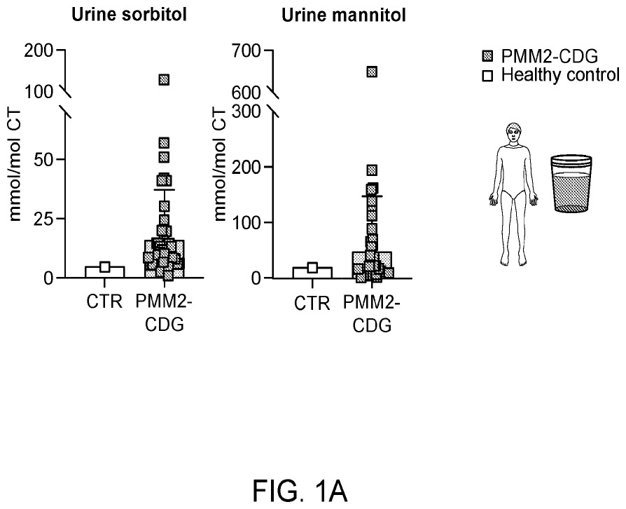

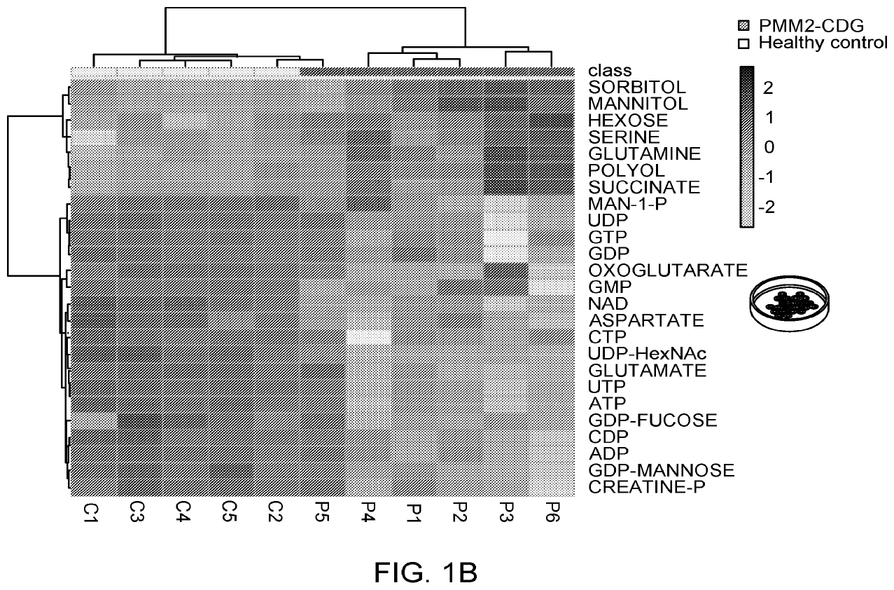

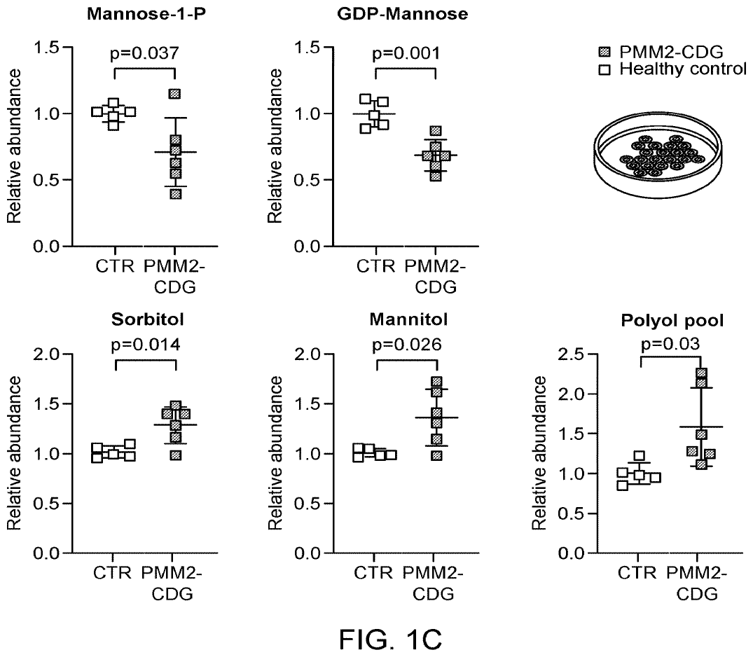

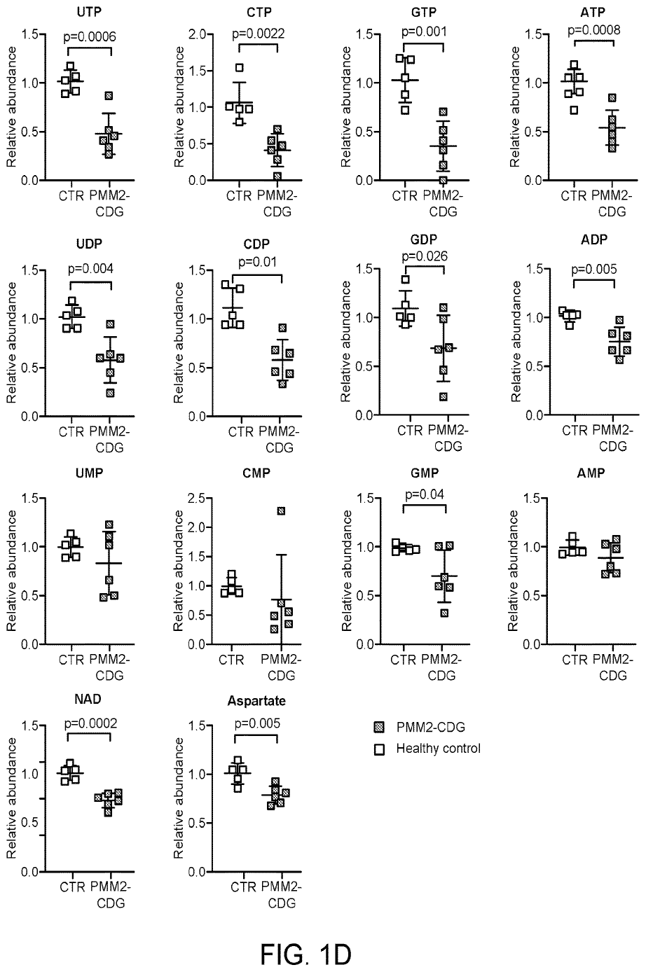

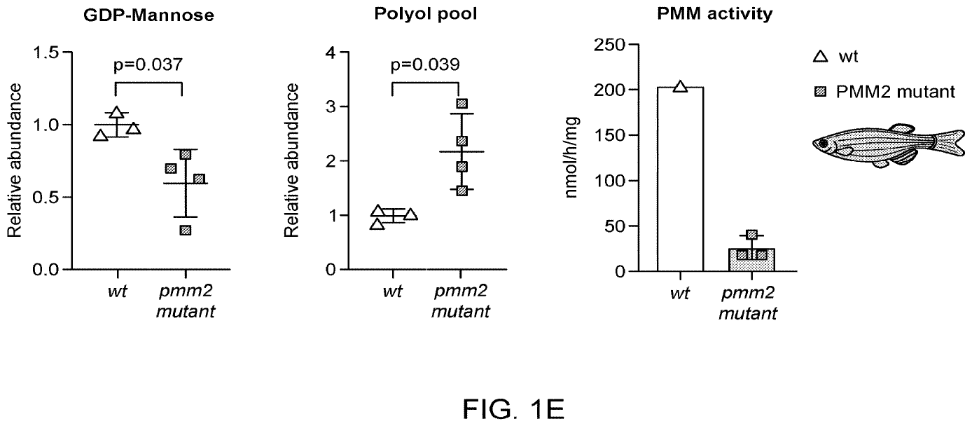

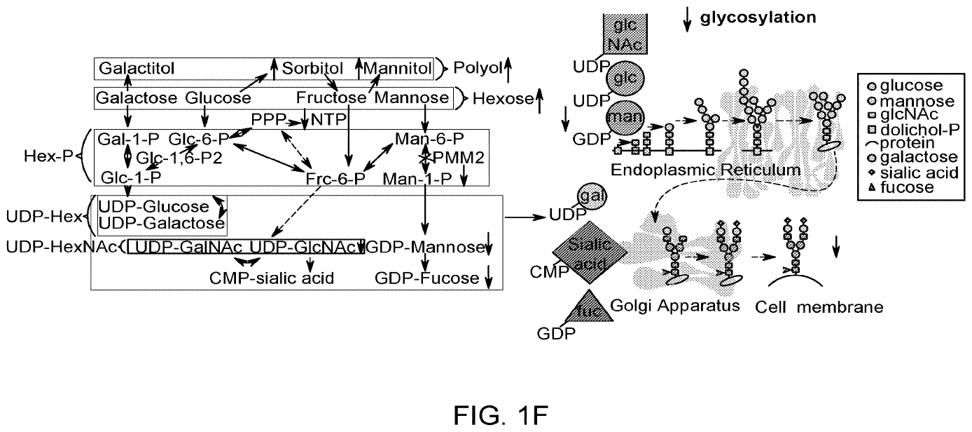

This study assessed urine polyol levels in 26 individuals with PMM2-CDG. Demographic and genetic characteristics of these individuals (P1-P50) and their urine polyol values are given in Table 2. In this cohort, 86% (43/50) of individuals with PMM2-CDG had elevated urine sorbitol (mean 16.3 mmol/mol creatinine; control reference range<5 mmol/mol creatinine), while 46% (23/50) had elevated urine mannitol (mean 50.18 mmol/mol creatinine; control reference range<20 mmol/mol creatinine) (FIG. 1A). These results confirm that abnormal polyol metabolism is common in PMM2-CDG.

### Global Metabolic Changes in PMM2-CDG Result in Increased Intracellular Polyols

Since PMM2-CDG does not commonly present with hyperglycemia, it was evaluated whether PMM2 deficiency resulted in altered glucose metabolism leading to an intracellular increase in polyols. To test this, and to assess global metabolic changes elicited by PMM2 deficiency, PMM2-CDG patient fibroblasts (P1-P6) with confirmed decreases in PMM enzymatic activity were metabolically profiled. Using high-throughput LC/MS metabolomics, over 45 metabolites covering major metabolic pathways (glycolysis, polyol metabolism, pentose phosphate pathway (PPP), nucleotide metabolism, hexosamine biosynthesis, TCA cycle, etc.) were simultaneously monitored in both PMM2-CDG and healthy fibroblasts, cultured in the presence of 5.5 mM (physiological) glucose.

Metabolomics analysis showed that PMM2-CDG fibroblasts clustered separately from healthy controls (FIG. 1B) due to significant differences in the abundances of multiple metabolites across two groups. Man-1-P and GDP-mannose were depleted in PMM2-CDG (FIG. 1B, C) corroborating the PMM2 deficiency. In contrast, polyol pools, specifically sorbitol and mannitol, were increased (FIG. 1B, C). In addition, the intracellular hexose pool (glucose, fructose, mannose, galactose) in PMM2-CDG fibroblasts was also increased (FIG. 1B). These results suggest an intracellular accumulation of hexoses upon PMM2 deficiency, which could explain the increased polyol production. Furthermore, changes in other pathways (glycolysis, TCA cycle) were noted (FIG. 1B) and it was found that the nucleotide-phosphate pools (NXP; NMP, NDP, NTP) were also depleted in PMM2-CDG fibroblasts (FIG. 1B, D). These findings suggest that PMM2 deficiency not only affects Man-1-P, GDP-mannose and glycosylation, but also results in broader metabolic effects.

The findings in fibroblasts were validated in the genetically stable pmm2^{m/m} (from here on referred to as pmm2 mutant) zebrafish. The pmm2 mutant zebrafish harbors a mutation in an essential splice site (pmm2^{sa1050}), which results in a hypomorphic pmm2 allele. By 6 days postfertilization (dpf) Pmm activity was significantly decreased in pmm2 mutant zebrafish embryos (FIG. 1E), with levels comparable to the enzymatic activity measured in PMM2-CDG patient fibroblasts. To determine if the metabolic changes observed in PMM2-CDG patient fibroblasts also occur in Pmm2-deficient zebrafish embryos, GDP-mannose and polyol pools were measured in whole lysates of 6 dpf pmm2 mutant and pmm2 wildtype (wt) zebrafish. Similar to the in vitro PMM2-CDG fibroblast model, GDP-mannose levels were reduced in pmm2 mutant embryos compared to wt (FIG. 1E). Polyols were also increased in pmm2 mutant embryos, further corroborating our findings from PMM2-CDG patient fibroblasts (FIG. 1E). It is important to note that zebrafish lysate contains both circulating (blood) and intracellular (tissue) metabolites. Therefore, the observed metabolites pools from the zebrafish experiments reflect both the intracellular and extracellular compartments.

In summary, these findings suggested that PMM2-CDG affects multiple biochemical pathways resulting in a decrease of GDP-mannose and an increase of intracellular polyols (FIG. 1F).

### Intracellular Polyols are Elevated in PMM2-CDG Regardless of the Environment

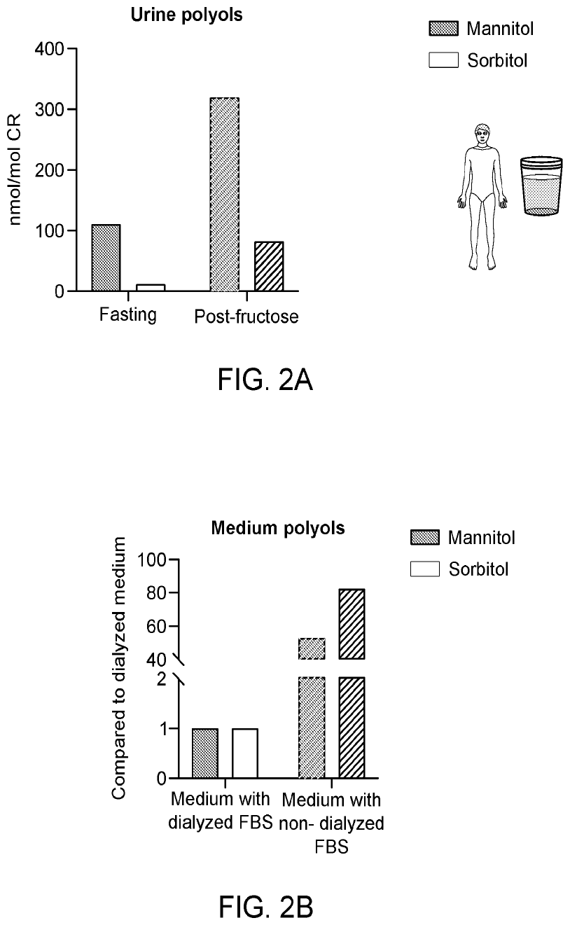

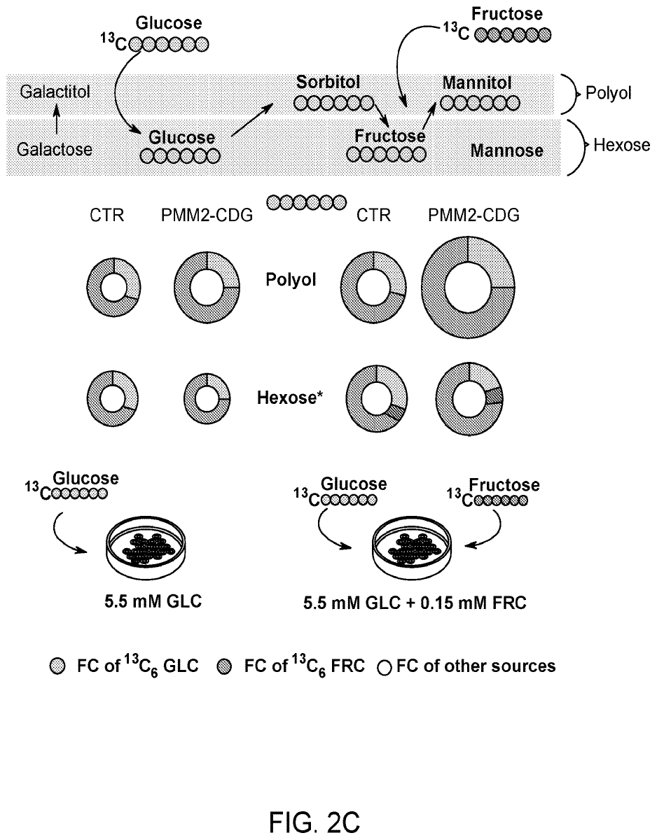

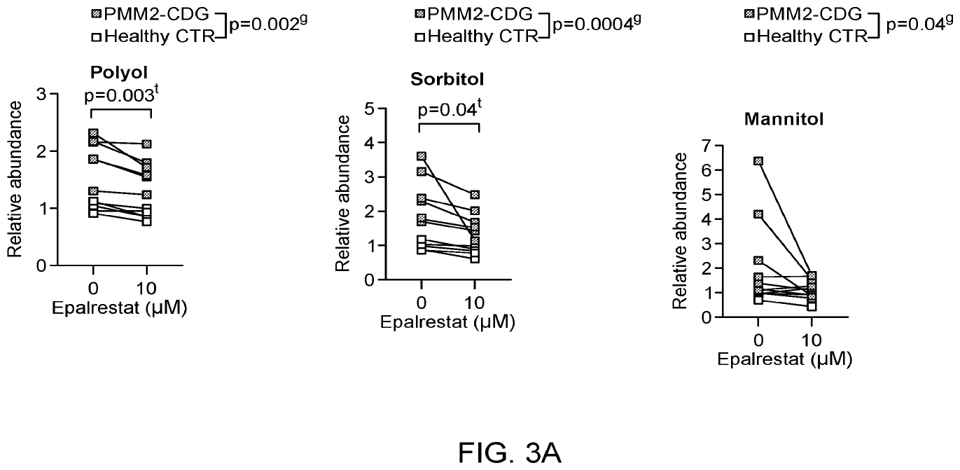

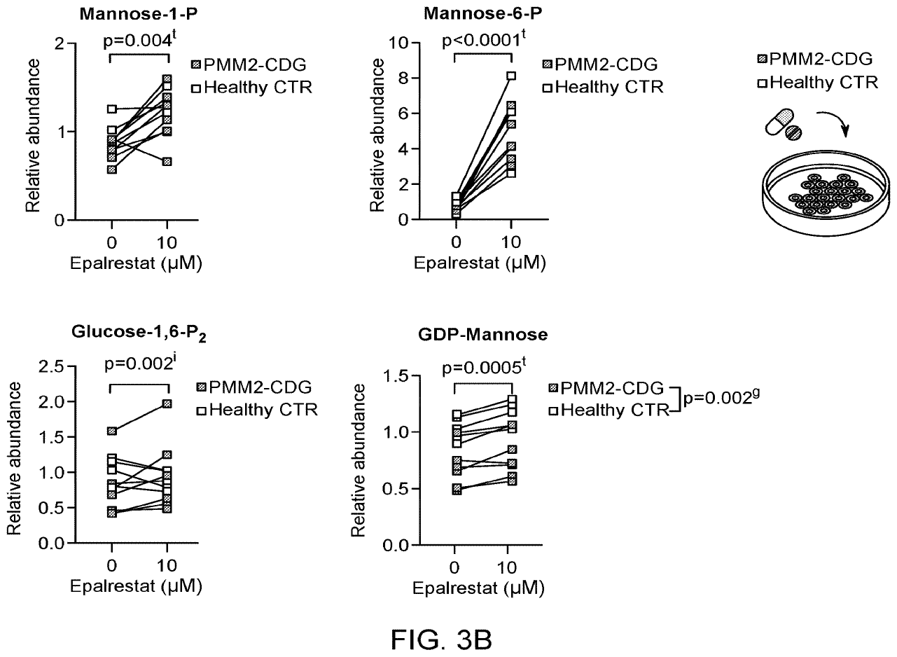

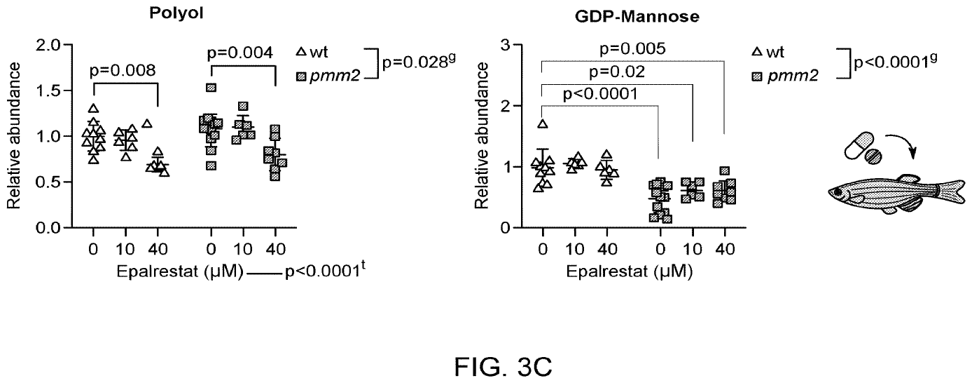

Since polyols were increased both intracellularly in PMM2-CDG fibroblasts and in the urine of PMM2-CDG patients (FIG. 1), it was evaluated whether the increased polyols were the result of environmental factors (such as e.g., diet, medication, medium used for culture of fibroblasts) or rather the direct consequence of the intracellular biochemical changes caused by PMM2 deficiency. First, to eliminate the possibility that increased intracellular polyols were caused by the presence and consequent uptake of polyols in the cell culture medium, the abundance of polyols in the standard medium containing non-dialyzed fetal bovine serum (FBS) was compared to the medium containing dialyzed FBS. It was found that non-dialyzed FBS contained 50-80 times more sorbitol and mannitol compared to the dialyzed FBS (FIG. 2A). The availability of polyols in the non-dialyzed medium could therefore resulted in the increase in polyols observed in the PMM2-CDG fibroblasts. To reduce the impact of polyols in the medium and test whether the increase in polyols was a result of intracellular changes due to PMM2 deficiency rather than increased uptake of polyols from the medium, dialyzed FBS was used in all subsequent experiments. It was found that using a medium with 5.5 mM (physiological) glucose and dialyzed FBS, polyols remained significantly increased in all 6 patient cell lines studied (FIG. 3A, B, C).

Next, the influence of dietary fructose on the urine polyol levels in PMM2-CDG was evaluated. Fructose has become one of the major sources of energy in the western diet and has been associated with numerous disorders such as diabetes, hypertension, obesity, etc. Peripheral fasting fructose levels range between 0.01-0.07 mM and can increase by 5-fold after consumption of a fructose rich meal. Fructose is likewise present in the normal diet of PMM2-CDG patients, who do not follow any special dietary regime, and increased fructose intake could result in increased urine polyol levels. The possibility of fructose contributing to the polyols increase seen in PMM2-CDG was therefore tested. First, a fructose tolerance test in individuals with PMM2-CDG (P5) was performed. The urine sorbitol increased approximately 7 times after the fructose tolerance test, while mannitol increased 3 times (FIG. 2B), indicating that dietary fructose could increase urine polyols in PMM2-CDG. Then, to investigate the contribution of fructose in intracellular polyol accumulation, patient and control fibroblasts were incubated in the presence of 5.5 mM glucose and 0.15 mM (average blood fructose concentration upon fructose rich meal)¹³C₆-fructose and medium containing dialyzed FBS (FIG. 2A). Addition of 0.15 mM ¹³C₆-fructose resulted in an increase in the polyol and hexose pool abundances as well as a 4% Fractional Contribution (FC) in total hexose pool in both PMM2-CDG and controls (FIG. 2C).

Together, these results suggest that the buildup of polyols in PMM2-CDG fibroblasts was caused by endogenous production, while the increased availability of fructose could contribute to the additional accumulation of polyols in PMM2-CDG and might have implications for the dietary regimen of individuals with PMM2-CDG.

Aldose Reductase Inhibition with Epalrestat Decreases Polyol Levels and Elicits Global Metabolic Changes

The finding of increased intracellular polyols in PMM2-CDG provided the first link between abnormal glycosylation and AR regulation. Next, the therapeutic mechanism of AR inhibition was assessed. The AR inhibitor epalrestat improved glycosylation in vitro and in vivo in PMM2-CDG (Ligezka et al, Ann. Neurol., 90(6):887-900 (2021)). It was therefore evaluated whether AR inhibition rewires the cellular metabolism by redirecting glucose away from the production of polyols towards other biochemical pathways, ultimately improving glycosylation.

To test this, PMM2-CDG and healthy fibroblasts grown in medium containing dialyzed FBS were treated with daily epalrestat (10 μM) supplementation. The concentration of epalrestat was previously established based on the pharmacological profile of epalrestat in the patient treated with 0.8 mg/kg/day TID (Ligezka et al, Ann. Neurol., 90(6):887-900 (2021)). The metabolomes of treated and non-treated cells were evaluated and compared.

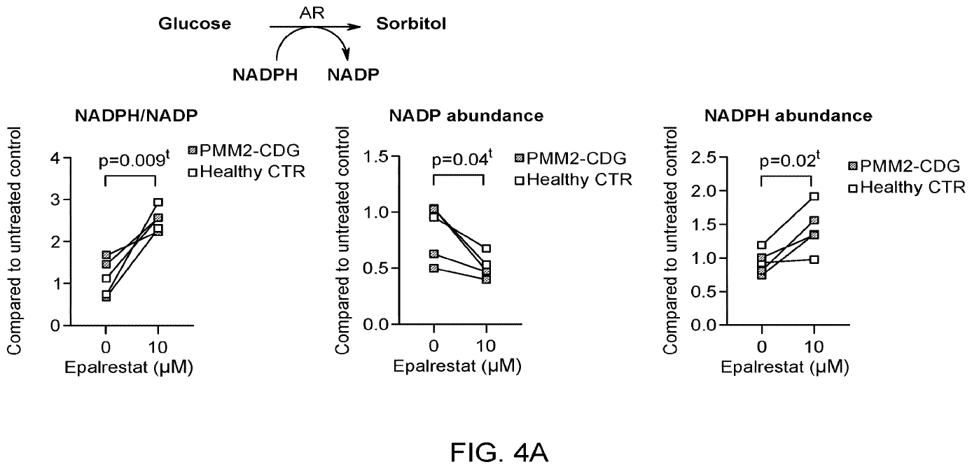

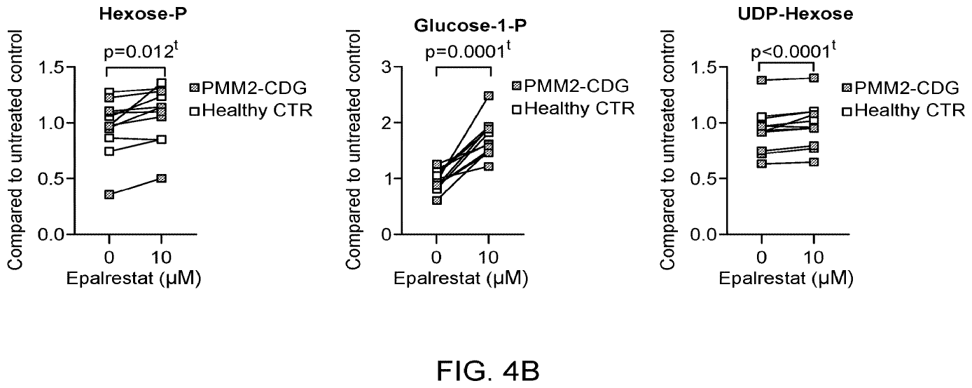

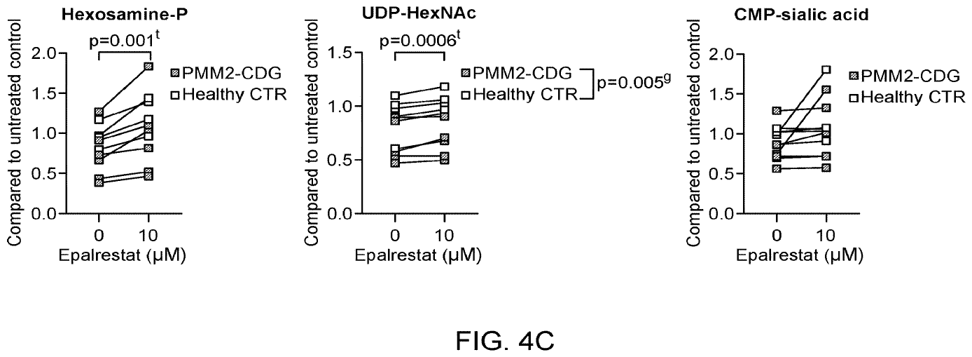

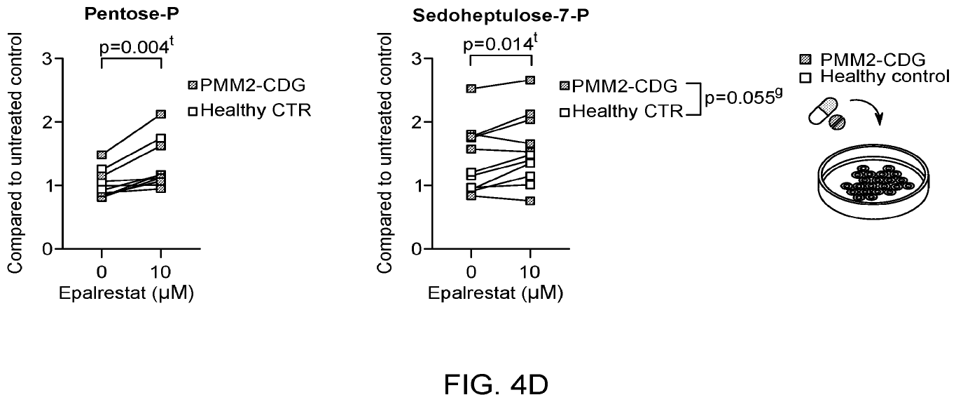

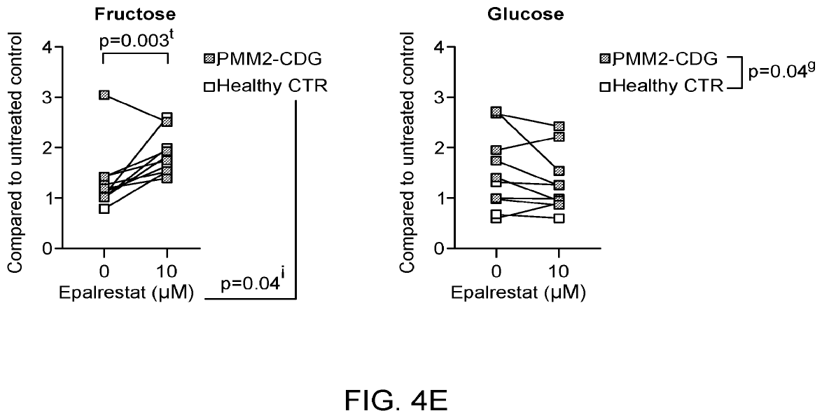

The results showed that epalrestat reduced the overall polyol pool in both patient and control cell lines (FIG. 3A). Specifically, sorbitol was significantly reduced following epalrestat treatment (FIG. 3A). Mannitol was also reduced in the majority of patient fibroblasts following epalrestat treatment, though its abundance fluctuated (FIG. 3A). It was also found that the NADPH/NADP ratio was increased upon epalrestat treatment, consistent with the inhibition of aldose reductase, which oxidizes NADPH to NADP (FIG. 4A).

Further, epalrestat treatment increased PMM2 related metabolites such as mannose-6-P (man-6-P), man-1-P, and GDP-mannose (FIG. 3B). The increase in man-1-P and GDP-mannose is crucial, as individuals with PMM2-CDG are unable to produce sufficient man-1-P and consequently GDP-mannose, which is necessary for correct glycosylation. These results also corroborated the findings of increased glycosylation in PMM2-CDG fibroblasts upon epalrestat treatment. Furthermore, the majority of patient fibroblasts showed decreased levels of glucose-1,6-P2 (Glc-1,6-P2) compared to the controls, and epalrestat increased the overall abundance of Glc-1,6-P2 in PMM2-CDG (FIG. 3B). Glc-1,6-P2 is an activator of PMM2 and is necessary for its mutase activity. As epalrestat may be a PMM2 activator, these data indicate that the increase in PMM enzymatic activity measured in patient fibroblasts could be mediated by the increase in Glc-1,6-P2.

Epalrestat treatment also resulted in changes in metabolite abundances across other biochemical pathways such as glycogen metabolism (hexose-P, glucose-1-P, UDP-hexose), the hexosamine biosynthesis pathway (hexosamine-P, UDP-GlcNAc, CMP-sialic acid), and the PPP (ribose-P, sedoheptulose-7-P) (FIG. 4), further supporting that aldose reductase inhibition results in global metabolic changes and shifts glucose metabolism away from polyol production towards (sugar) nucleotide production, which is beneficial in PMM2-CDG.

### Effects of Aldose Inhibition are Corroborated In Vivo in Pmm2 Mutant Zebrafish

To corroborate these findings in vivo, 5 dpf wt and pmm2 mutant zebrafish embryos were treated with a single dose of either 10 μM or 40 μM epalrestat for 24 hours. The decreased level of GDP-mannose measured in untreated pmm2 mutant embryos was increased upon epalrestat supplementation, while polyol levels were reduced compared to untreated pmm2 mutant zebrafish (FIG. 3C).

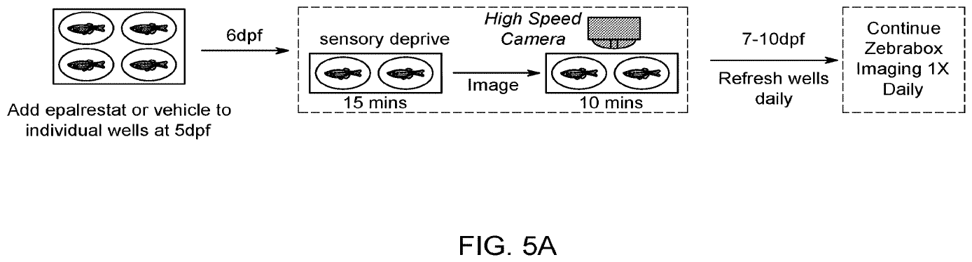

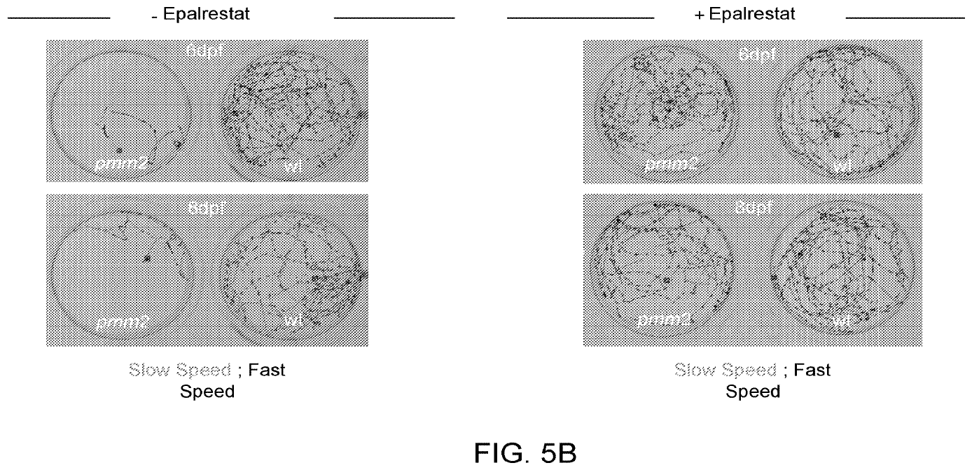

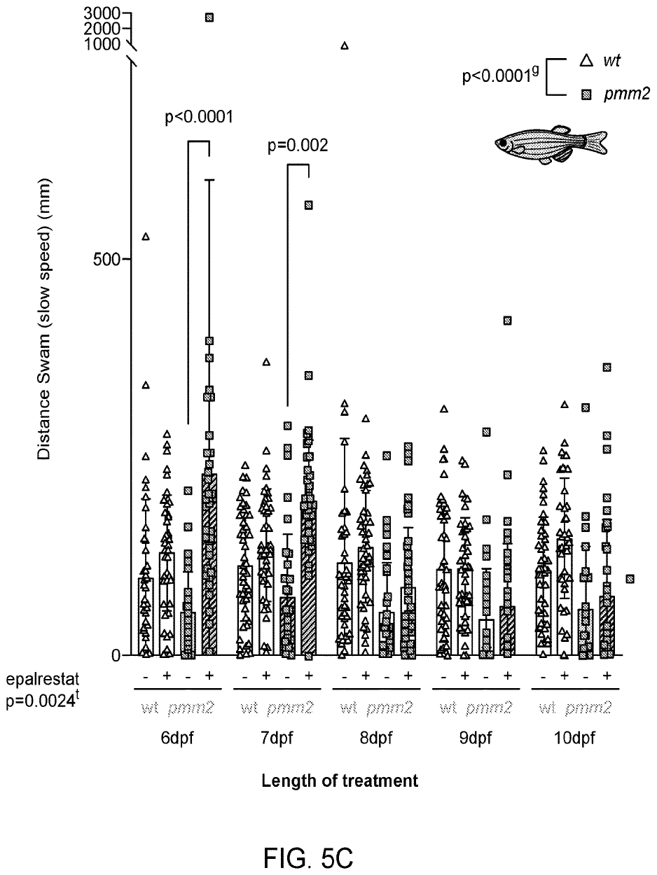

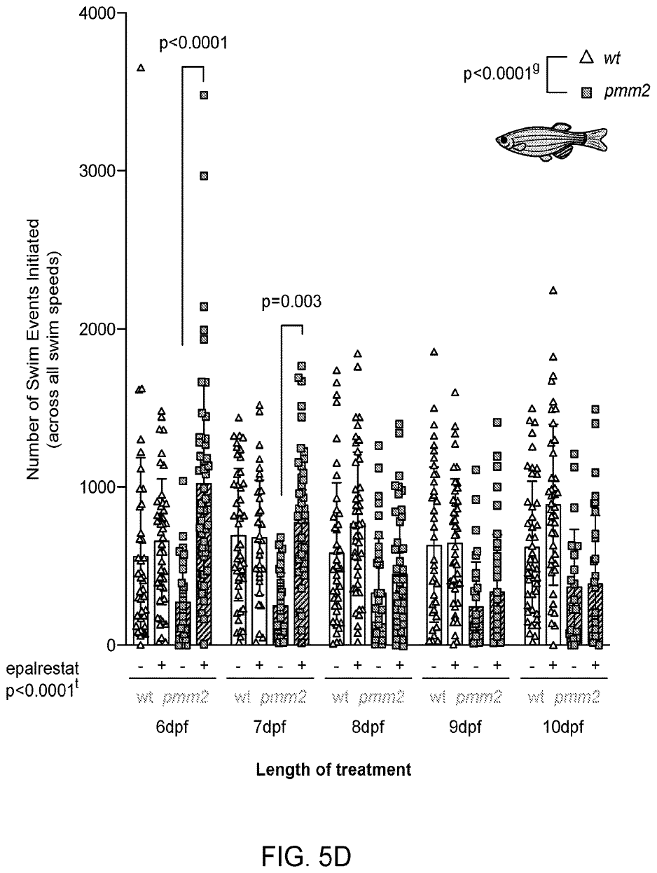

Using an automated behavioral tracking system (FIG. 5A, B), it was evaluated if epalrestat treatment improved the previously characterized defects in pmm2 zebrafish swim behavior. To answer this, wt and pmm2 mutant zebrafish were treated with 10 μM daily epalrestat, which resulted in no improvement in swimming behavior. Then, both wt and pmm2 mutant zebrafish were treated with 20 M daily epalrestat, which increased the distance swam and the number of swim events initiated in the pmm2 mutant zebrafish (FIG. 5 C,D). The improvement was most significant at 6 dpf and 7 dpf (FIG. 5 C,D). Moreover, no significant difference in the viability between treated and untreated pmm2 mutant zebrafish was found.

### Combination of Epalrestat and Nucleoside Supplementation as a Therapeutic Option for CDG

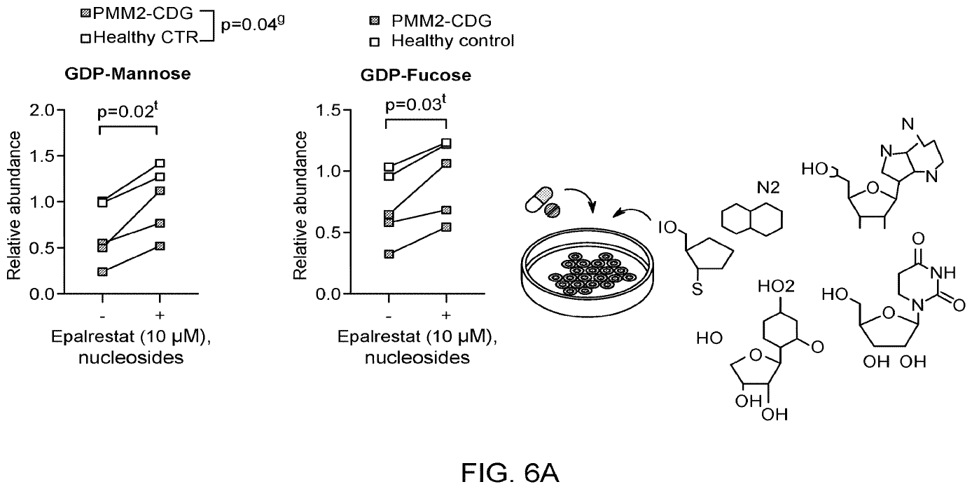

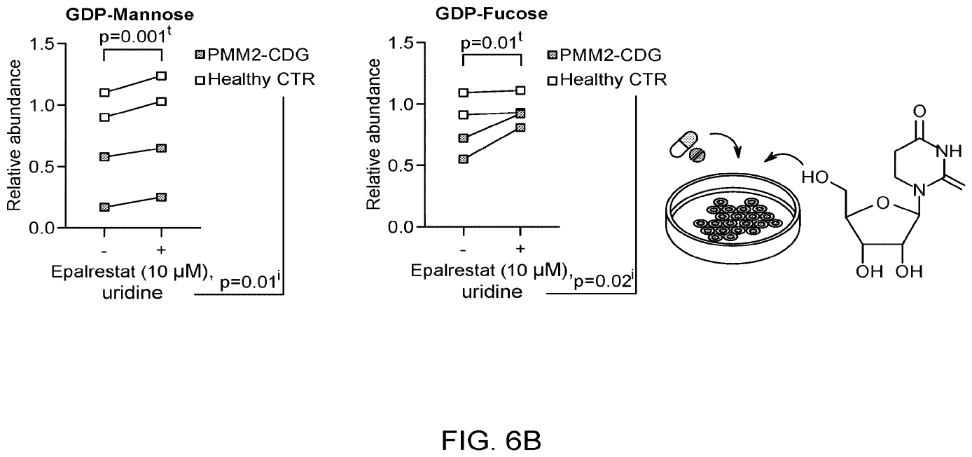

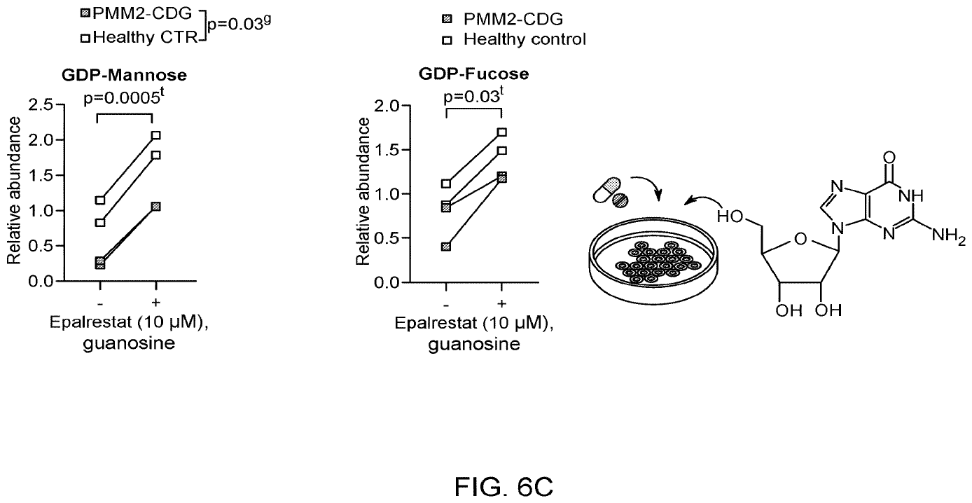

Though an overall increase in sugar nucleotides was noticed after epalrestat treatment, GDP-mannose levels in patient fibroblasts never reached the same levels as the ones seen in controls. As it was found that the nucleotide levels in PMM2-CDG were also depleted (FIG. 1), it was tested whether the addition of nucleosides (the combination of guanosine, uridine, adenosine, thymidine, cytidine etc.), or uridine or guanosine (30 μM each) alone, to epalrestat could restore sugar nucleotide pools. The abundance of GDP-mannose further increased after the combination therapy of epalrestat and nucleosides (FIG. 6A) as well as that of uridine and epalrestat (FIG. 6B). Finally, it was found that addition of 30 μM guanosine in combination with epalrestat restored GDP-mannose and GDP-fucose abundance comparable to levels seen in healthy controls (FIG. 6C).

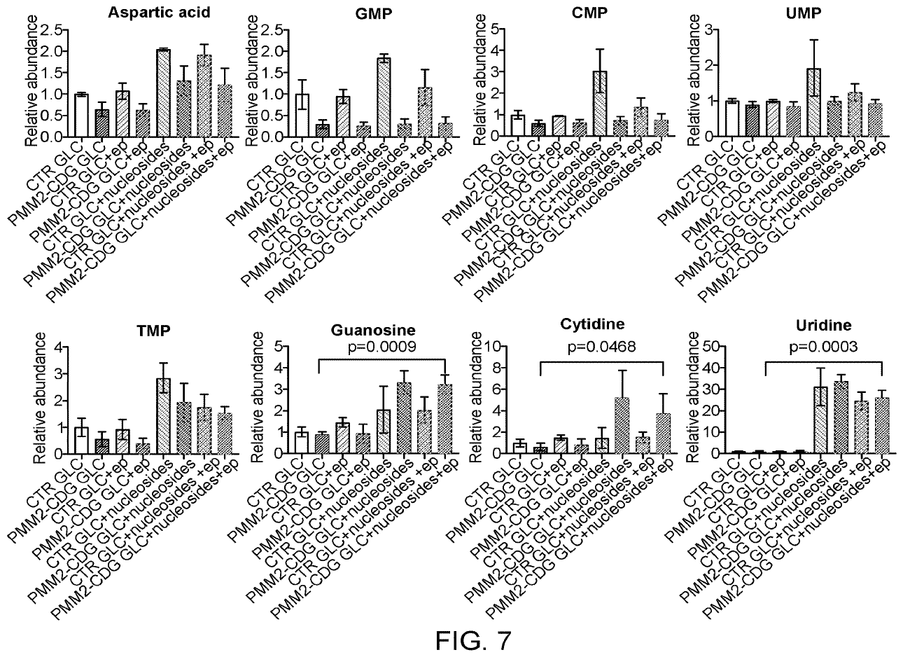

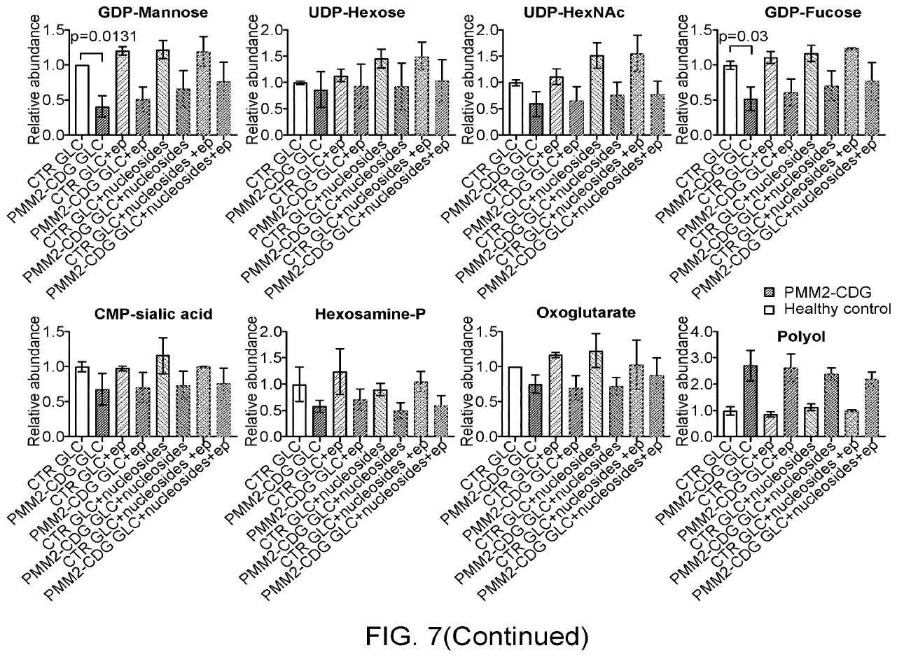

Interestingly, guanosine, cytidine and uridine abundances were higher in patients upon nucleoside supplementation, while their corresponding nucleotide monophosphates (NMPs) were lower (FIG. 7). These data indicate there might be a block in the formation of NMPs in PMM2-CDG affected fibroblasts.

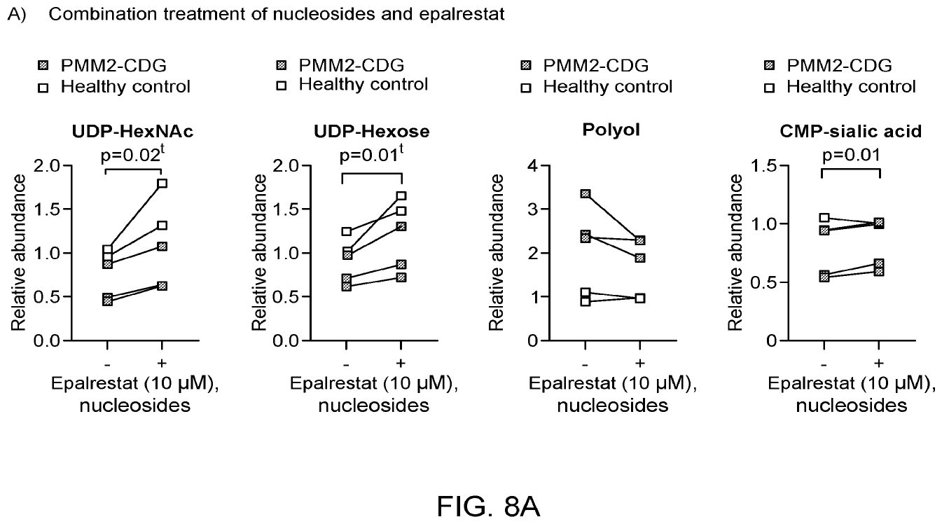

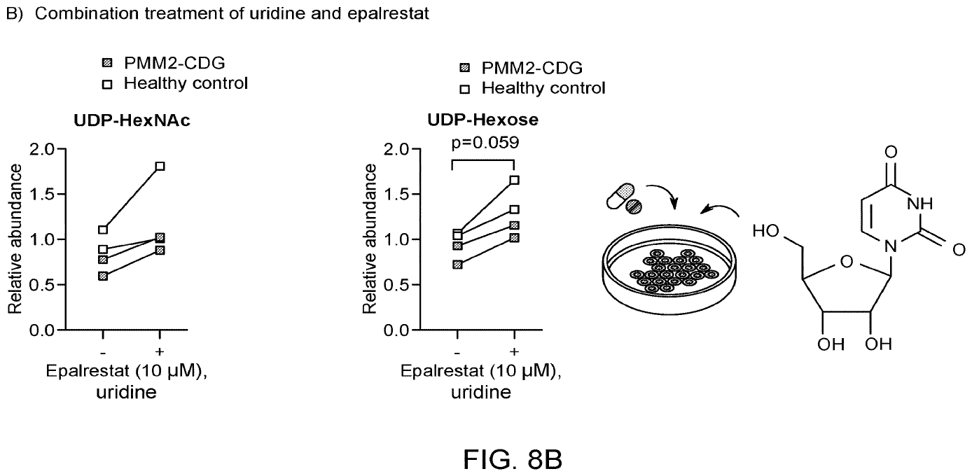

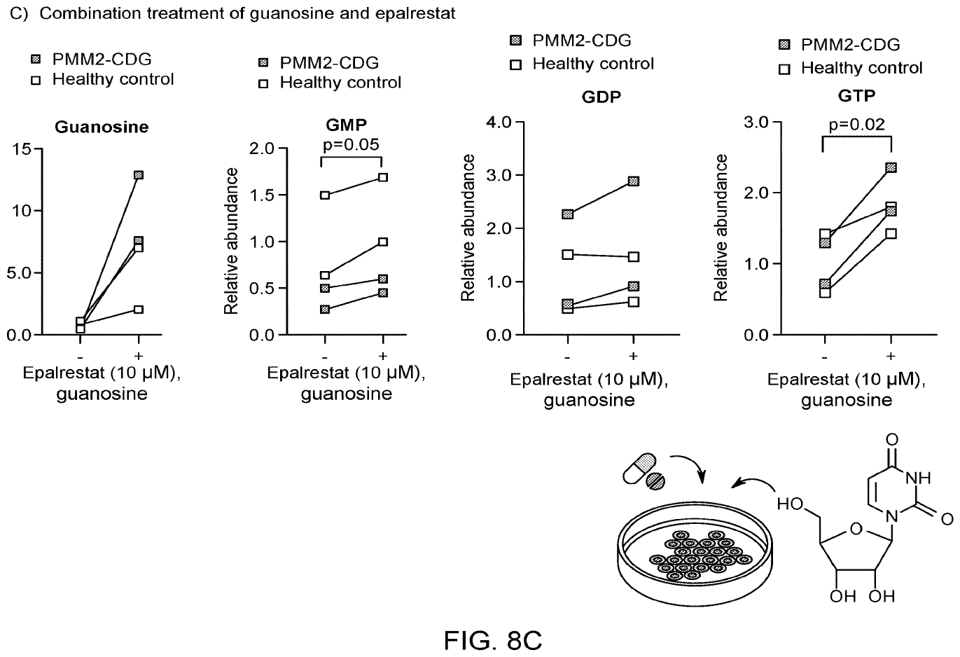

In addition, the levels of NMPs were lower in the samples treated with both nucleotides and epalrestat compared to the samples treated with nucleotides alone, while the levels of sugar nucleotides were the highest. These data suggest that supplemented nucleotides were possibly being used to build sugar-nucleotides in a higher extent when coupled with epalrestat (FIG. 6, 7). Further, it was found that aspartic acid, as well as other metabolites relevant to PMM2-CDG were also increased upon nucleotide supplementation (FIG. 8).

Therefore, these results demonstrate that PMM2-CDG patients could additionally benefit from a combination therapy of epalrestat and nucleosides, specifically guanosine.

### Tracer Studies Reveal AR Inhibition Rewires Glucose Metabolism Resulting in Increased De Novo (Sugar) Nucleotide Synthesis and Glycosylation

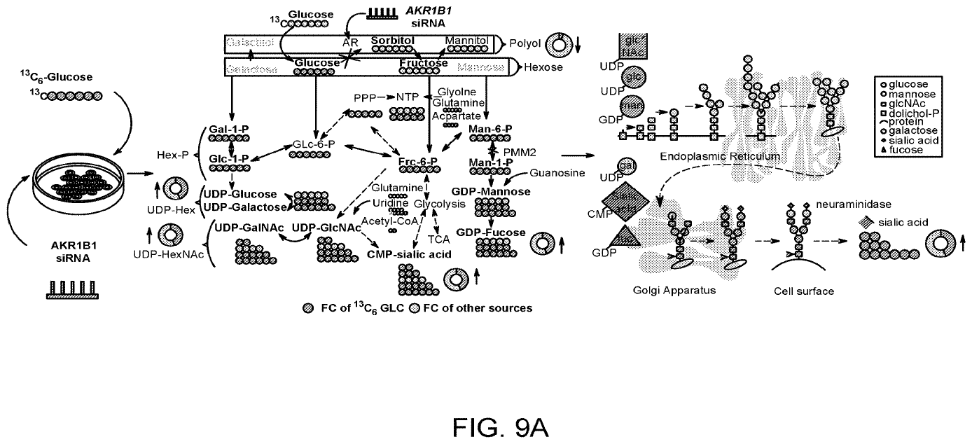

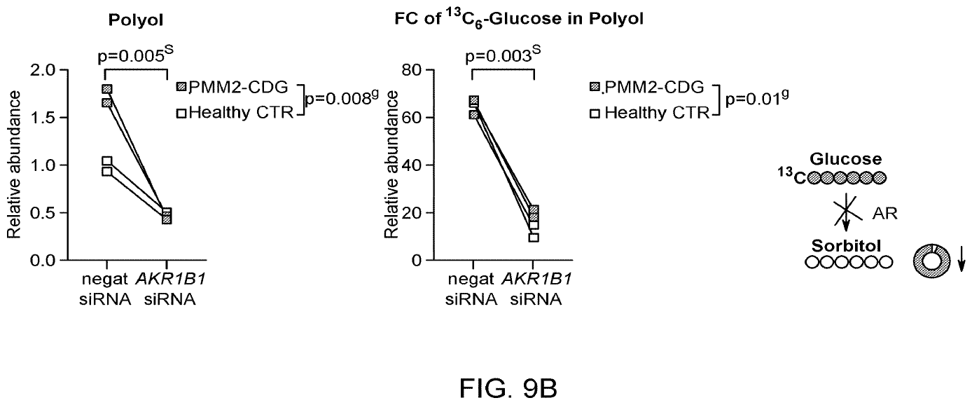

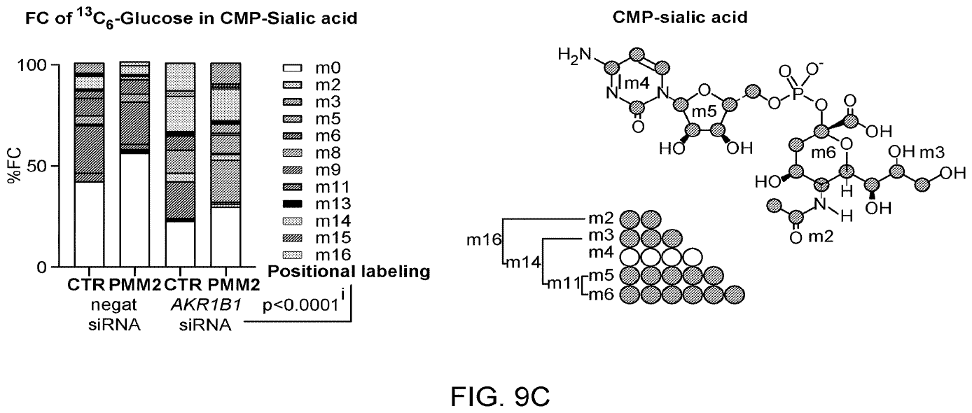

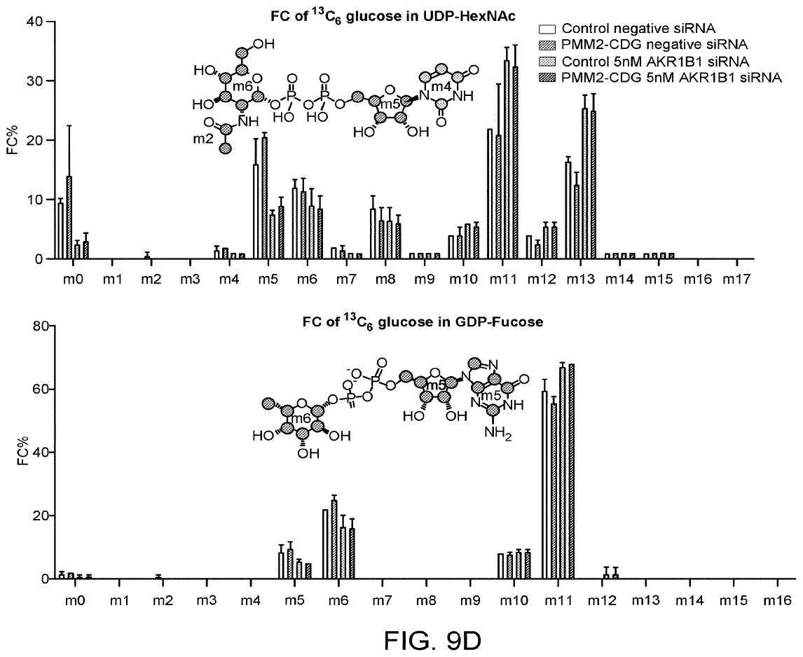

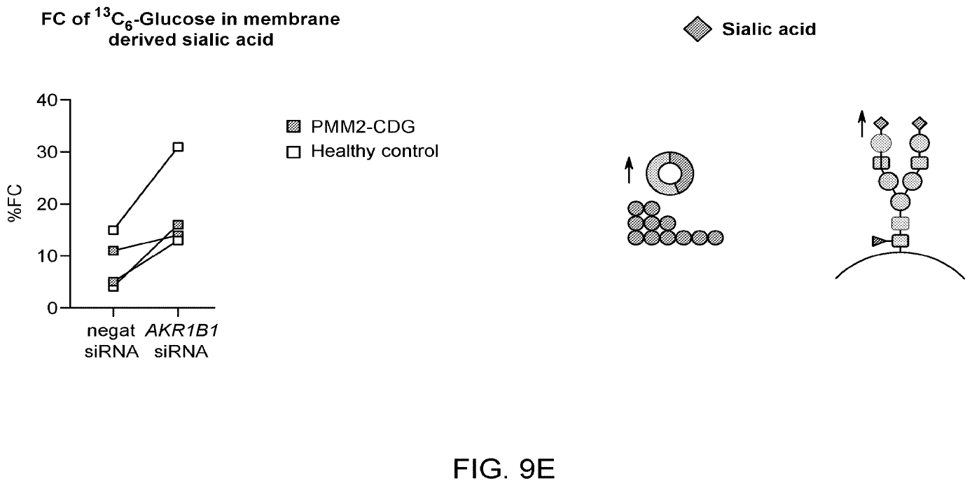

To further elucidate the link between aldose reductase and glycosylation and to confirm that the therapeutic effect of epalrestat is through the inhibition of the aldose reductase (AR) and not an off-target effect, tracer experiments following the siRNA inhibition of AKR1B1 (AR gene) were performed (FIG. 9A). It was tested whether siRNA mediated knock-down (KD) of AKR1B1 would result in a more explicit effect than the one seen with the chemical inhibition by epalrestat, which is a non-competitive inhibitor of AR.

While the changes in the abundances of metabolites do not clearly depict the directionality of their pathways (an increase in a metabolite can both indicate an increased flux upstream of the metabolite in question as well as a decrease in the flux downstream of the metabolite in question), the changes in the fractional contribution (FC) of ¹³C₆-glucose provide direct insight into the activities of pathways relying on it.

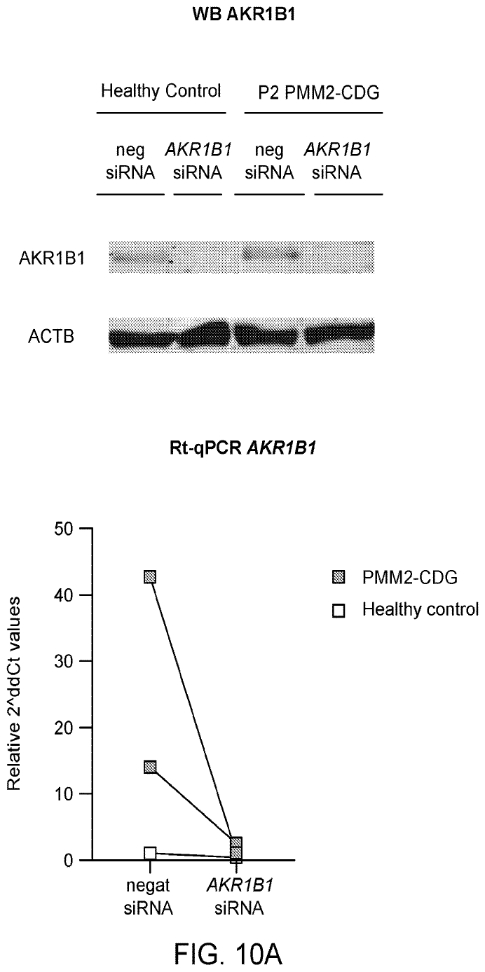

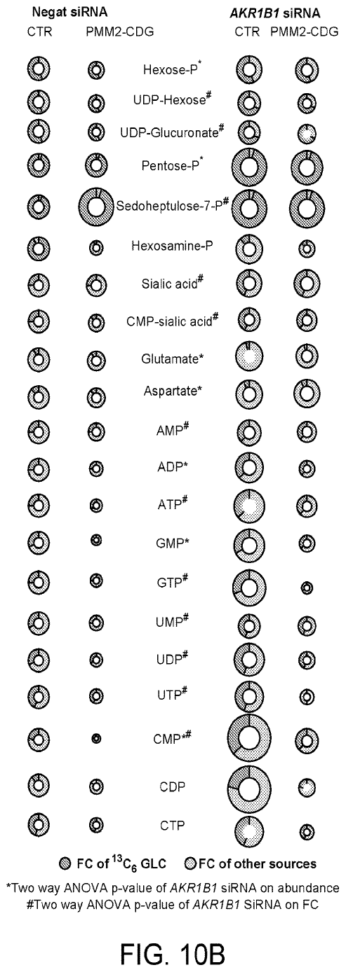

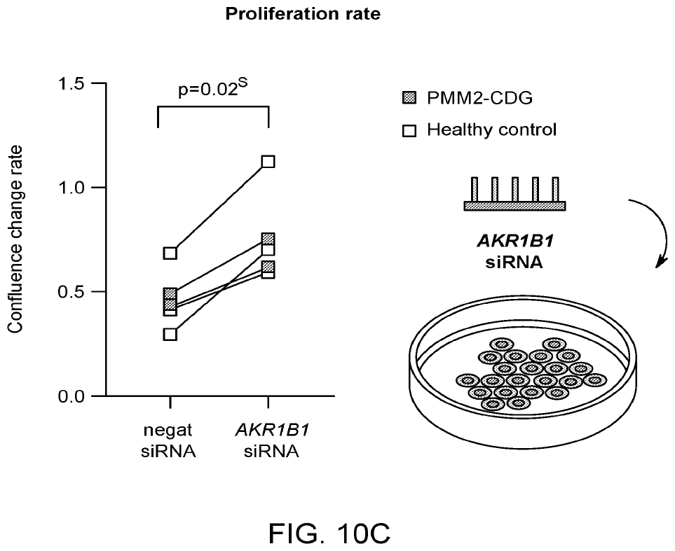

Following siRNA inhibition of AKR1B1—confirmed by RT-qPCR and WB (FIG. 26A)—significant changes in FC of ¹³C₆-glucose were observed in several metabolites, implying that glucose flux was significantly altered upon AR inhibition in both patient and control fibroblasts (FIG. 9A, FIG. 10B).

The abundance of polyols and the FC of ¹³C₆-glucose in polyols were significantly decreased upon AKR1B1 KD (FIG. 9B), verifying that glucose conversion to sorbitol was drastically inhibited (FIG. 9B). Apart from the reduction in polyol synthesis metabolism, AKR1B1 KD resulted in a significant increase in both the FC of ¹³C₆-glucose and metabolite abundance in (sugar) nucleotides (FIG. 10B), which suggested de novo (sugar) nucleotide synthesis was promoted upon AR inhibition. Since de novo nucleotide synthesis occurs during intensive proliferation, the effect of AR inhibition on proliferation rate of the cells was also assessed. It was found that AR inhibition increased proliferation in both control and patient fibroblasts (FIG. 10C), corroborating the tracer experiment findings.

To further investigate the impact of AKR1B1 KD on pathway activity, the isotopologues of CMP-sialic acid were assessed (FIG. 9C). CMP-sialic acid is an important glycosylation building block and is synthetized through the combination of several moieties coming from different biochemical pathways such as the five-carbon ribose (from PPP), six-carbon hexose (from fructose-6-P), two-carbon acetyl group (from acetyl-CoA), and a three-carbon group (from phospho-enolpyruvate (PEP)). Therefore, the positional labeling—the percentage of labeled carbons ranging from m0 (no carbons labeled) to m20 (all carbons labeled)—can provide information about the activities of the pathways providing those moieties. Upon AKR1B1 KD, an increase in m11, m14 and m16 of CMP-sialic was noted while m5, m6 and m8 decreased (FIG. 9C). These data imply an increased flux of glucose going towards glycolysis (m3 labeled moiety from PEP), acetyl-CoA production (m2 labeled moiety) and de novo nucleotide synthesis (m11=m5 labeled moiety of ribose+m6 labeled moiety of hexose) (FIG. 9C). Similar changes in positional labeling was seen in other nucleotide sugars (e.g UDP-HexNAc, GDP-Fucose) (FIG. 9D).

The increase of CMP-sialic acid abundance and FC of ¹³C₆-glucose indicated that AR inhibition promoted de novo synthesis of sugar nucleotides, sugar donors of glycans. Sialic acid, the terminal sugar capping the ends of the mature N-glycans, is transferred from CMP-sialic acid to the terminus of correctly assembled glycan chains. This however did not provide sufficient information to conclude that AKR1B1 KD also results in increased glycosylation. Therefore, it was evaluated whether the observed changes in glucose flux upon AR inhibition would also be reflected in improved glycosylation. To answer this, the cell membranes containing mature glycans were isolated and the terminal sialic acid was enzymatically removed upon incubation with sialidase (N-acetylneuraminidase), which was then subjected to LC/MS. By specifically assessing the isotopologues of sialic acid in analogy with CMP-sialic acid, information on the changing glucose fluxes was retrieved. In this regard, FC of ¹³C₆-glucose in the sialic acid generated from membrane bound glycans increased significantly, suggesting that AR inhibition led to an increase in sialylation (FIG. 9E). These data also corroborate the finding that AR inhibition leads to overall glycosylation improvement as assessed by glycoproteomics in PMM2-CDG fibroblasts treated with epalrestat.

### Off/Label Epalrestat Treatment Improves Clinical Outcome Measures in PMM2-CDG Patients

Epalrestat was trialed in three pediatric PMM2-CDG patients (P5-7) to assess long-term treatment safety and efficacy. No side-effects were observed. Escalating doses of epalrestat were well-tolerated. All laboratory values collected at baseline and intermittent results during the epalrestat treatment are given in Table 2.

TABLE 2
Off-label epalrestat treatment improves clinical outcome measures in PMM2-CDG
patients. Clinical and biochemical findings of patients at baseline, and intermittent results
during treatment with epalrestat.

| Patient | P5 | | | | P6 | | | P7 | |
|---|---|---|---|---|---|---|---|---|---|
| Age*/Sex | 9/F | | | | 8/M | | | 3/F | |
| PMM2 pathogenic variants | c.422G > A, c.415G > A | | | | c.422G > A, c.548T > C | | | c.422G > A, c.647A > T | |
| Parameter\Time point (months) | Baseline | 12 | 24 | 28 | Baseline | 12 | 18 | Baseline | 12 |
| Epalrestat dose (mg/kg day) | 0.8 | 1.5 | 2 | 3 | 0.8 | 1.5 | 3 | 0.5; 0.8$ | 1.5 |
| Total NPCRS (max 78) | 24 | 20 | 18 | 18 | 19 | 19 | 18 | 18 | 15 |
| Modified BARS (max 54) | 39 | 29 | 33 | 30 | 45 | 29 | 27 | N/A | N/A |
| Goal attainment scale (scale −2 −+ 2) | | | | | | | | | |
| Goal 1⁺ | −2 | −1 | −1 | N/A | −2 | −1 | N/A | −2 | −2 |
| Goal 2⁺ | −2 | −1 | −1 | N/A | −1 | −1 | N/A | −2 | −1 |
| Goal 3⁺ | −2 | −2 | −2 | N/A | −2 | −2 | N/A | −2 | −1 |

Laboratory investigations

| | | | | | | | | | |
|---|---|---|---|---|---|---|---|---|---|
| AST U/L (C: < 50 U/l) | 48 | 37 | 34 | 40 | 58 | 40 | 38 | 548 | 80 |
| ALT U/L (C < 45 U/l) | 23 | 24 | 25 | 25 | 67 | 32 | 49 | 997 | 105 |
| AT III | 0.88 | 0.99 | 0.97 | 103 | 0.25 | — | 0.39 | 0.21 | 0.37 |
| (C: 0.80-1.30 IU/mL) | | | | | | | | | |
| CDT mono-oligo /di-oligo | 0.09 | 0.06 | 0.09 | 0.07 | 0.69 | 0.43 | 0.47 | 1.60# | 1.58 |
| ratio (C < 0.06) | | | | | | | | | |
| CDT a-oligo/di-oligo | 0.005 | 0.003 | 0.010 | 0.08 | 0.196 | 0.111 | 0.105 | 0.919# | 1.033 |
| ratio (C < 0.011) | | | | | | | | | |
| Urine Sorbitol | 19.9 | 10.7 | 7.5 | 7.86 | 50.80# | 12.81 | 9 | 9.85 | 0.03 |
| (<5 mmol/ mol Cr) | | | | | | | | | |
| Urine Mannitol | 648.6 | 32.6 | 45.3 | 93.19 | 23.90# | 14.77 | 47 | 10.37 | 1.89 |
| (< 20 mmol/mol Cr) | | | | | | | | | |

Abnormal values are marked in bold.
Abbreviations: ALT = Alanine transaminase, AST = Aspartate transaminase, ATIII = antithrombin III, BARS- = Brief Ataxia Rating Scale; CDT = Carbohydrate Deficient Transferrin; Cr = Creatinine; C = control; NPCRS = Nijmegen Pediatric CDG Rating Scale; m = months; N/A = not applicable; — = value not reported.
*age at the time of publication (years);
⁺see methods for goals description;
#first available results 3 months after starting treatment;
^{$}patient was first started on 0.5 mg/kg/day, and after a month switched to 0.8 mg/kg/day.

Due to the multisystemic PMM2-CDG phenotype (liver function abnormalities, coagulation abnormalities, developmental delay, neurologic involvement, ataxia etc.), the beneficial effects of epalrestat were assessed in relation to several aspects of the PMM2-CDG phenotype. Safety parameters (complete blood cell count, liver transaminases, bilirubin, alkaline phosphatase, prothrombin time, international normalized ratio), growth, and vital signs were monitored throughout the treatment.

Treatment with epalrestat decreased urine polyol levels which remained mostly stable during treatment. A fluctuation in mannitol in P5 was noted, which underwent a fructose loading test (FIG. 2B). Furthermore, improvement in biochemical markers which are commonly abnormal in PMM2-CDG, such as liver transaminase levels, coagulation parameters (ATIII), and carbohydrate deficient transferrin (CDT; clinical glycosylation marker) were observed. At baseline, CDT was abnormal to a different degree in all three patients. Normalization of CDT was seen in P5. P6 showed significant improvement in CDT with a steady trend towards normalization at 18 months of therapy and escalating doses of epalrestat. Baseline CDT results of P7 were not available, and the first result was obtained after 3 months of therapy with epalrestat. However, at the one-year mark, a slight improvement was seen in CDT. Moreover, the declining modified Brief Ataxia Rating Scale (BARS) scores indicated improvement in ataxia (P5, P6). Although P7 was not yet eligible for the BARS assessment due to her age, improvement in lower body strength and speech was observed on epalrestat. Phenotype Severity was assessed by Nijmegen Pediatric CDG Rating Scale (NPCRS), and improvement was noted in 2 out of 3 patients (P5, P7). Additionally, all patients self-reported clinical improvement and quality of life improvement during epalrestat treatment as assessed by goal attainment scale (GAS).

### Example 2: Treating PM2-CDG

A human identified as having PMM2-CDG is administered one or more AR inhibitors (e.g., epalrestat) and one or more nucleosides (e.g., guanosine). For example, a juvenile human is administered a composition including epalrestat and guanosine. The administered one or more AR inhibitors (e.g., epalrestat) and one or more nucleosides (e.g., guanosine) can increase the level of PMM2-related metabolites within the mammal.

### Example 3: Treating PAM2-CDG

A human identified as having PMM2-CDG is administered one or more AR inhibitors (e.g., epalrestat) and one or more nucleosides (e.g., guanosine). For example, a juvenile human is administered a composition including epalrestat and guanosine. The administered one or more AR inhibitors (e.g., epalrestat) and one or more nucleosides (e.g., guanosine) can reduce the severity of one or more symptoms of PMM2-CDG.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal having a congenital disorder of glycosylation (CDG), wherein said method comprises administering, to said mammal, (a) one or more aldose reductase (AR) inhibitors and (b) one or more nucleosides and/or one or more nucleotides, wherein one or more symptoms of said CDG are reduced.

2. The method of claim 1, wherein said mammal is a human.

3. (canceled)

4. The method of claim 1, wherein said human is from about 1 month to about 17 years of age.

5. The method of claim 1, wherein said mammal is identified as having said CDG.

6. The method of claim 1, wherein said CDG is a phosphomannomutase 2 (PMM2)-CDG.

7. The method of claim 1, wherein said one or more AR inhibitors are selected from the group consisting of epalrestat, AT007, fidarestat, sorbinil, salfredin b11, and any combination thereof.

8. The method of claim 1, wherein said one or more nucleosides are selected from the group consisting of guanosine, uridine, adenosine, thymidine, cytidine, and any combination thereof.

9. The method of claim 1, wherein said method comprises administering epalrestat and guanosine.

10. The method of claim 1, wherein said one or more AR inhibitors and said one or more nucleosides are administered, to said mammal, by oral administration.

11-13. (canceled)

14. The method of claim 1, wherein said one or more symptoms are selected from the group consisting of ataxia, speech delay, seizures, global developmental delay, intellectual disability, coagulation abnormalities, and liver abnormalities.

15. A method for increasing a level of one or more PMM2-related metabolites within a mammal, wherein said method comprises administering, to said mammal, (a) one or more AR inhibitors and (b) one or more nucleosides and/or one or more nucleotides.

16. The method of claim 15, wherein said mammal is a human.

17-18. (canceled)

19. The method of claim 15, wherein said mammal has a CDG.

20. The method of claim 15, wherein said CDG is a PMM2-CDG.

21. The method of claim 15, wherein said one or more AR inhibitors are selected from the group consisting of epalrestat, AT007, fidarestat, sorbinil, salfredin b11, and any combination thereof.

22. The method of claim 15, wherein said one or more nucleosides are selected from the group consisting of guanosine, uridine, adenosine, thymidine, cytidine, and any combination thereof.

23. The method of claim 15, wherein said method comprises administering epalrestat and guanosine to said mammal.

24. The method of claim 15, wherein said one or more AR inhibitors and said one or more nucleosides are administered, to said mammal, by oral administration.

25-27. (canceled)

28. The method of claim 15, wherein said one or more PMM2-related metabolites are selected from the group consisting of mannose-1-phophate (man-1-P), mannose-6-phosphate (man-6-P), glucose-1,6-diphosphate (Glc-1,6-P2), guanosine, uridine, adenosine, thymidine, cytidine, guanosine triphosphate (GTP), guanosine diphosphate (GDP), GDP-mannose, GDP-fucose, UDP-hexose, UDP-HexNAc, and any combination thereof.

29. (canceled)

30. A composition comprising (a) one or more AR inhibitors and (b) one or more nucleosides and/or one or more nucleotides.

31. (canceled)
